(19) United States
(12) Patent Application Publication
(10) Pub. No.: US 2025/0340875 A1
(43) Pub. Date: Nov. 6, 2025

(54) SIRNA MOLECULE AGAINST HUMAN TENASCIN-C (TNC) AND A PHARMACEUTICAL COMPOSITION COMPRISING IT

(71) Applicant: MEDICOFARMA BIOTECH SA, Warsaw (PL)

(72) Inventors: Katarzyna Monika ROLLE, Kamionki (PL); Dariusz Józef WAWRZYNIAK, Poznan (PL); Michal Julian PRENDECKI, Poznan (PL); Malgorzata GRABOWSKA, Poznan (PL); Aleksandra Joanna GRZANKA, Poznan (PL); Daria Magdalena STUBE, Niechanowo (PL); Agnieszka Marta CHABOWSKA-KITA, Poznan (PL); Lukasz Leszek PRZYBYL, Chyby (PL)

(21) Appl. No.: 18/866,276

(22) PCT Filed: May 17, 2023
(86) PCT No.: PCT/PL2023/050034

§ 371 Date: Nov. 15, 2024

(30) Foreign Application Priority Data
May 17, 2022 (PL) P.441204
May 17, 2022 (PL) P.441205

(51) Int. Cl. C12N 15/113; A61P 35/00

(52) U.S. Cl. CPC C12N 15/113; A61P 35/00; C12N 2310/14; C12N 2310/321; C12N 2310/322; C12N 2310/3515; C12N 2310/3519; C12N 2320/31

(57) ABSTRACT

The invention relates to siRNA molecules against the human tenascin-C (TNC) transcript sequence for silencing of TNC expression. The invention relates also to pharmaceutical compositions comprising said siRNA molecules and mixtures thereof, and use said compositions in therapy and/or prevention of the development of cancer characterized by increased TNC expression in a human by inhibiting TNC expression. In particular, said cancer is selected from glioma, breast cancer, ovarian cancer, and pancreatic cancer.

The present invention relates to siRNA molecules against the human tenascin-C (TNC) transcript sequence, pharmaceutical compositions comprising said siRNAs and mixtures thereof, and use of said compositions in a therapy and/or prevention of the development of cancer characterized by increased TNC expression in a human by inhibiting TNC expression. In particular, said cancer is selected from glioma, breast cancer, ovarian cancer, and pancreatic cancer.

### TECHNICAL FIELD

Tenascin-C (TNC) is an extracellular matrix glycoprotein affecting adhesion, invasion, migration and proliferation of tumour cells, which has been used, for example, in brain tumour therapy (glioblastoma multiforme, GBM). An RNA interference (RNAi) approach was used to inhibit TNC [Zukiel et al. 2006; Rolle et al. 2010; Barciszewski et al. 2007].

TNC is highly expressed in the cancer tissue of most malignant tumours involving the brain [Leins et al. 2003] and ovaries [Wilson et al. 1996], in some breast [Jahkola et al. 1998; Guttery et al. 2010] and pancreatic [Liot et al. 2021] cancers. A high level of TNC positively correlates with the degree of malignancy of the tumour—more malignant cancers usually present higher level of TNC, which is an unfavourable prognostic marker. In tumour tissue, TNC is mainly found in the extracellular matrix of the fibrous stroma of highly malignant cancers including colon and breast cancer, fibrosarcomas, lung cancers, melanomas, squamous cell carcinoma, bladder tumour, prostate adenocarcinoma and along the tumour margin [Chiquet-Ehrismann et al. 2003]. Decidedly higher levels of TNC have been observed in GBM homogenates than in normal brain [Pas et al. 2006]. High level of TNC expression in human gliomas and astrocytomas, also correlate with enhanced angiogenesis [Behrem et al. 2005; Jallo et al. 1997]. Furthermore, TNC is cancer stem cells marker in GBM [Nie et al. 2015].

### BACKGROUND ART

The publication EP2121927B1 discloses a method to inhibit malignant glioma using a double-stranded RNA (dsRNA), called ATN-RNA, which comprises a fragment of the TNC mRNA sequence with nucleotides 405 to 567. The dsRNA molecule used is naked, delivered in the presence of calcium ions and thus susceptible to very rapid degradation by, inter alia, RNA hydrolysing enzymes. The assumption of this therapy does not describe the possibility of using this molecule in other cancer types.

TNC inhibition with a radiolabelled monoclonal antibody administered directly into the locus after the removed tumour in combination with radiotherapy and adjuvant chemotherapy significantly increases the survival of patients with primary GBM [Reardon et al. 2002; Reardon et al. 2003]. Similarly, the administration of a double-stranded RNA fragment with 163 base pairs in length, ATN-RNA, directly into the locus after the removed tumour effectively inhibits the growth of both primary and recurrent human brain tumours through inhibition of TNC synthesis and significantly increases survival and quality of life of patients after glioma resection [Zukiel et al. 2006; Rolle et al. 2010; Wyszko et al. 2008]. Importantly, the applied therapy does not exhibit any pro-inflammatory effects in primary human GBM cells and in vitro glioma cell lines [Rolle et al. 2010]. Multifactor analysis exhibited that ATN-RNA effectively reduces the size of recurrent brain tumours, suggesting that inhibition of TNC expression is particularly important for inoperable tumours [Wyszko et al. 2008]. The reduction in TNC level resulting from in vitro application of ATN-RNA resulted in a decrease in cancer cell migration [Grabowska et al. 2019]. In the cited study, the ATN-RNA molecule was added to cancer cells in complex with magnetic nanoparticles coated with polyethyleneimine (PEI). A similar approach based on the delivery of siRNA to cells in complex with PEI was used for silencing the growth factor PTN which inhibited the proliferation of glioma cancer cells in vitro and tumour growth in a mouse xenograft model without inducing significant immunogenicity [Grzelinski et al. 2006].

In a mouse model of human glioblastoma multiforme, resulting from intracranial in-oculation of U87MG-Luc cells in mice, simultaneous silencing of transcripts of genes encoding epithelial growth factor receptor (EGFR) and TNC significantly inhibited the proliferation of cancer cells [Fu et al. 2021]. The study describes a method of preparation of a genetic system that, after entering the recipient organism, provides the instructions for the production of siRNA molecules in the liver. The in vivo-produced siRNA molecules are then packaged into microvesicles and secreted into the bloodstream, from where they reach the target organ, where they can regulate the levels of EGFR and TNC transcripts. The publication does not address the delivery of finished siRNA molecules with anti-cancer potential, thus presenting a different approach to that described by the authors of the present application. Despite the use of a specific guiding sequence, the level of siRNA molecules in the brain was more than 100 times lower than in the liver, which may potentially have a negative impact on the efficiency and safety of the therapy and speaks in advantage of local administration of siRNA molecules directly to the affected area. Importantly, the anti-TNC sequence presented by the authors of the publication targets a different fragment compared to the sequences presented in the present application.

The publication WO2011107586A1 relates to a method for treating brain cancer in a subject by administering a therapeutically effective amount of a modulator of the in-teraction between SPARC-related modular calcium binding 1 (SMOC1) and TNC, for example, a specific antibody or a siRNA. However, no experimental data confirming silencing, no siRNA sequence against TNC and no details of lipid carriers are provided in said application.

Silencing the TNC gene transcript with ATN-RNA significantly inhibits the proliferation, migration and differentiation of human cells and multicellular breast cancer spheroids in vitro and has no immunogenic effect [Wawrzyniak et al. 2020]. In addition, down-regulation of TNC transcript level with short hairpin-forming RNA (shRNA) reduces breast tumour growth and increases the efficiency of immunotherapy used to treat triple-negative breast cancer (TNBC) in a mouse xenograft model of human breast cancer [Li et al. 2020]. TNC secreted by human breast cancer cells is positively correlated with the occurrence of lung [Oskarsson et al. 2011], liver [Ma et al. 2012] and lymph node [Yang et al. 2017] metastases. A meta-analysis exhibited that high TNC level correlates with cancer stage and poor prognosis for patients in 14 cancer types, including breast cancer [Ming et al. 2019].

The stroma of malignant ovarian tumours is characterized by a high level of TNC compared with benign tumours [Wilson et al. 1996]. TNC is mainly secreted by fibroblasts and plays an important role in the invasion of cancer cells by affecting their adhesion and migration in vitro [Wilson et al. 1999]. Furthermore, TNC level in the serum of patients suffering from epithelial ovarian cancer is significantly higher compared to healthy individuals [Tas et al. 2016].

Pancreatic adenocarcinoma (PAAD), is one of the most common and aggressive forms of pancreatic cancer and one of the most common causes of death from cancer disease in humans worldwide. Given the poor efficacy of current therapeutic methods in the treatment of pancreatic cancer, which is associated with rapid metastasis and patient death, new treatment methods for this cancer disease are urgently needed. TNC level is relatively low in the normal pancreas, but increases significantly in cancer cells and is positively correlated with pancreatic cancer progression [Esposito et al. 2006; Balasenthil et al. 2011; Cai et al. 2018]. Determination of TNC and tissue factor inhibitor levels in patients' plasma enables the diagnosis of early-stage PAAD, improves the diagnostic efficacy of the existing biomarker CA 19-9, and allows differentiation between patients with pancreatitis, PAAD and diabetes [Balasenthil et al. 2017].

TNC level increase with cancer cell invasiveness and shRNA-mediated reduction of TNC expression results in significant inhibition of pancreatic cancer cell proliferation and migration in vitro [Qian et al. 2019]. Similarly, siRNA-mediated reduction of TNC gene transcript level through modulation of the cell cycle process significantly reduces the proliferation of human pancreatic cancer cells in vitro and in a mouse xenograft model [Cai et al. 2018].

There are known examples of the use of lipid nanoparticles (LNPs) as a carrier for the delivery of nucleic acids, e.g. in plasmid form, to glioma cells in the context of research [Yoshida et al. 2004]. In addition, LNPs with potential use in the treatment of glioma have been disclosed, e.g. as carriers for cytostatic drugs [Ortega-Berlanga et al. 2021]. In contrast, the drug patisiran, an siRNA complex with LNPs [Hu et al. 2020], and vaccines in the form of an mRNA complex with LNPs against coronavirus (SARS-CoV-2) (e.g. Moderna, CureVac, BioNTech) are used in clinical practice [Dammes et al. 2020]. LNP systems as nucleic acid carriers are complex structures (about 100 nm), usually consisting of aminolipids as the main component (ionizable-MC3, KC2 or cationic—DOTAP), phosphatidylcholine lipids, cholesterol and polyethylene glycol-lipid conjugate (PEG-lipid). Various types of LNPs are known. Cationic lipids are one of the more commonly described LNPs as carriers facilitating the penetration of nucleic acids into cells. At the same time, neutral (ionizable) LNPs exhibited similar effects [Halder et al. 2006], while having less immunogenicity [Chon et al. 1991] and better penetration in the tumour-like microenvironment [Lieleg et al. 2009].

The development of ionizable lipids was one of the major steps in the development of LNP technology. Lipids of this type are neutrally loaded at physiological pH and acquire a positive charge at acidic pH, allowing a pH-dependent electrostatic in-teraction with negatively charged nucleic acid in the external environment. When transported into the cell, ionizable LNPs become ionized in low pH environments, for example endosomes and lysosomes, leading to the disruption of the complex and the release of the charge in the cell [Schlich et al. 2021]. Such hybrid properties increase the half-life of the complexes in the peripheral blood and facilitate the release of the complex content in the target cells, which provides an advantage over cationic lipids and is particularly important for intravenous administration of the preparation [Semple et al. 2001]. An example is the ionizable lipid Dlin-MC3-DMA, which has a pKa of 6.44 and is used in an approved preparation of patisiran (Onpattro®, Alnylam Pharma-ceuticals, Cambridge, MA, USA) [Akinc et al. 2019].

Traditional methods for producing LNPs involve forming a lipid film followed by hydration of the film with an aqueous buffer comprising nucleic acid to passively envelop the load [MacLachlan et al. 2007]. This typically leads to large (>100 nm) and heterogeneous particles with low encapsulation efficiency that require the addition of a size reduction technique for example extrusion or sonication. The sonication and extrusion, which are necessary for this preparation method, negatively affect the stability of the nucleic acids [Furusawaa et al. 2014]. In addition, this method is difficult to scale up and achieve reproducibility of the produced LNPs—a necessary condition for the approval of a specific substance as a drug in therapy. Synthesis of LNPs by microfluidic mixing allows for scalable, reproducible and rapid preparation of carriers with specific size (<100 nm) and physicochemical properties (i.e. polydispersity index (<0.25)) depending on the lipids, buffers and type of nucleic acid used [Roces et al. 2020].

The publication U.S. Pat. No. 8,598,333B2 relates to a siRNA molecule chemically synthesized to reduce a transcript level of the Eg5 gene highly expressed in cancers. The possibility of modifying the above-mentioned siRNA molecule is also claimed e.g. by adding a 2′O methyl group, and a pharmaceutical composition that comprises an siRNA molecule with a cationic lipid and a non-cationic lipid, wherein the composition of the lipid nanoparticles is not indicated.

The publication EP2241323A1 relates to the use of tenascin-W (TNW) lowering siRNA molecules in the treatment of brain cancers, including astrocytoma, glioma and oligodendroglioma. Unlike TNC, TNW expression in gliomas is limited to blood vessels and does not occur directly in cancer cells [Martina et al. 2010], so TNC silencing will alter other molecular mechanisms than lowering the TNW level. EP2241323A1 does not disclose experimental data or details of the siRNA sequence for anticancer use.

### SUMMARY OF INVENTION

Despite the previously described solutions and knowledge of TNC, which is highly expressed in cancer tissues of most malignant tumours, including brain, breast, ovarian and pancreatic tumours, and whose expression increases in cancer tissues as they grow, effective means and methods for the therapy of said cancers are among the unmet medical needs. There is, therefore, a need to provide a new technology using more selective molecular tools with better results in influencing cancers, the development of which is correlated with an increase in TNC level, particularly for the treatment of glioma, ovarian, breast and pancreatic cancer, based on molecular tools that will at the same time give better therapeutic results, be easier to deliver, less immunogenic and less toxic to the patient, and easier to store and administer, which will translate into the availability of the therapy and it's going beyond experimental therapy. It is also important that the technology is as versatile as possible and applicable to different types of cancer, including glioma, breast cancer, ovarian cancer or pancreatic cancer. Equally important is the form—the carrier—in which such improved targeted molecular tools will be delivered, so that they are effective in therapy, easy and reproducible to generate and have as few side effects as possible.

Thus, there is still a need for a technology that can be used as a therapeutic approach to inhibit the infiltration processes of brain cancers, especially gliomas that cannot be effectively removed surgically. Such a therapeutic approach is the use of TNC synthesis inhibition for the suppression of human brain tumours by using the developed siRNA-ATN and siRNA-TNC according to the invention.

Due to the poor efficacy of existing therapeutic methods for the treatment of cancer, which is associated with rapid metastasis and death of the patient, new means for the treatment of this disease are urgently needed which are proposed in the present invention.

The state of the art does not disclose or suggest siRNA sequences that are active molecules formed within the known therapeutic molecule ATN-RNA and that can effectively have an inhibitory effect on the development of several types of cancer including glioma, breast cancer, ovarian cancer or pancreatic cancer, without demonstrating the disadvantages and side effects that the known ATN-RNA molecule has, in particular its immunogenicity or toxicity to all cells.

The carriers of siRNA molecules according to the invention can be lipid nanoparticles (LNPs), in particular either cationic or ionizable lipids. Examples of such nanoparticles are lipid carriers comprising, for example, cationic lipids (i.e. DOTAP, DOTMA, 18PA), ionizable lipids (i.e. DLin-KC2-DMA, DLin-MC3-DMA, DLin-DMA, DODMA, DODAP), cholesterol, pegylated lipids (i.e. DMG-PEG, DSPE-PEG), auxiliary lipids (phospholipids i.e. DPPC, DOPE, DSPC).

The application of the microfluidic mixing technique for the production of lipid complexes with siRNA according to the invention for the therapy of glioma, breast cancer, ovarian cancer, pancreatic cancer, and highly expressing TNCs, solves a number of problems described above, e.g. it allows the elimination of additional synthesis steps, i.e. sonication and extrusion, which negatively affect the stability of nucleic acids, thus increasing the biological efficiency in the therapy of administered lipid nanoparticle complexes with siRNA. In addition, the lipid complexes used, obtained by microfluidic mixing techniques, provide siRNA-ATN and siRNA-TNC with proven selective effects on target cancers (glioma, breast cancer, ovarian cancer, pancreatic cancer).

In addition, the use of overhangs (especially 2 nt in length), increases the efficiency of silencing of the target transcript by the siRNA. This is because the PAZ domain of the AGO2 protein interacts with single-stranded, overhanging RNA fragments. In addition, in a siRNA hybrid having a single overhang [e.g. dTdT], the overhanging strand is selected as the effector strand, further improving the target TNC silencing by the siRNA according to the invention.

The invention thus relates to an siRNA molecule against a human TNC transcript sequence (disclosed in the NCBI database as NM_002160.4, Seq ID No: 25) for silencing TNC expression which (i) is at least 80% identical with the complementary sequence of the TNC mRNA in the 423-443 nucleotide region of Seq ID No: 25, and/or comprises a molecule with a sequence at least 80% identical with Seq ID No: 3; (ii) is at least 80% identical with the complementary sequence of the TNC mRNA in the 451-471 nucleotide region of Seq ID No: 25, and/or comprises a molecule with a sequence at least 80% identical with Seq ID No: 5; (iii) is at least 80% identical with the complementary sequence of the TNC mRNA in the 472-492 nucleotide region of Seq ID No: 25, and/or comprises a molecule with a sequence at least 80% identical with Seq ID No: 7; (iv) is at least 80% identical with the complementary sequence of the TNC mRNA in the 495-515 nucleotide region of Seq ID No: 25, and/or comprises a molecule with a sequence at least 80% identical with Seq ID No: 9; (v) is at least 80% identical with the complementary sequence of the TNC mRNA in the 538-558 nucleotide region of Seq ID No: 25, and/or comprises a molecule with a sequence at least 80% identical with Seq ID No: 11; (vi) is at least 80% identical with the complementary sequence of the TNC mRNA in the 4625-4645 nucleotide region of Seq ID No: 25, and/or comprises a molecule with a sequence at least 80% identical with Seq ID No: 13.

Preferably, the siRNA molecule (i) is at least 85%, more preferably 90%, more preferably 95% identical with the complementary sequence of the TNC mRNA in the 423-443 nucleotide region of Seq ID No: 25, and/or comprises a molecule at least 85%, more preferably 90%, more preferably 95% identical with Seq ID No: 3; (ii) is at least 85%, more preferably 90%, more preferably 95% identical with the complementary sequence of the TNC mRNA in the 451-471 nucleotide region of Seq ID No: 25, and/or comprises a molecule at least 85%, more preferably 90%, more preferably 95% identical with Seq ID No: 5; (iii) is at least 85%, more preferably 90%, more preferably 95% identical with the complementary sequence of the TNC mRNA in the 472-492 nucleotide region of Seq ID No: 25, and/or comprises a molecule at least 85%, more preferably 90%, more preferably 95% identical with Seq ID No: 7; (iv) is at least 85%, more preferably 90%, more preferably 95% identical with the complementary sequence of the TNC mRNA in the 495-515 nucleotide region of Seq ID No: 25, and/or comprises a molecule at least 85%, more preferably 90%, more preferably 95% identical with Seq ID No: 9; (v) is at least 85%, more preferably 90%, more preferably 95% identical with the complementary sequence of the TNC mRNA in the 538-558 nucleotide region of Seq ID No: 25, and/or comprises a molecule at least 85%, more preferably 90%, more preferably 95% identical with Seq ID No: 11; (vi) is at least 85%, more preferably 90%, more preferably 95% identical with the complementary sequence of the TNC mRNA in the 4625-4645 nucleotide region of Seq ID No: 25, and/or comprises a molecule at least 85%, more preferably 90%, more preferably 95% identical with Seq ID No: 13.

A preferred siRNA molecule comprises a sequence of at least 21 nucleotides in length.

A preferred siRNA molecule comprises a sequence of at least 21 nucleotides in length, preferably between 21-30 nucleotides, more preferably 21-27 nucleotides, wherein (i) is at least 85%, more preferably 96%, more preferably 99%, more preferably 100% identical with a sequence complementary to TNC mRNA in the 423-443 nucleotide region of Seq ID No: 25, and/or comprises a molecule at least 85%, more preferably 90%, more preferably 96%, more preferably 99%, more preferably 100% identical with Seq ID No: 3, most preferably is a molecule with the sequence of Seq ID No: 3; (ii) is at least 85%, more preferably 96%, more preferably 99%, more preferably 100% identical with the complementary sequence of the TNC mRNA in the 451-471 nucleotide region of Seq ID No: 25, and/or comprises a molecule at least 85%, more preferably 90%, more preferably 96%, more preferably 99%, more preferably 100% identical with Seq ID No: 5; most preferably is a molecule with the sequence of Seq ID No: 5; (iii) is at least 85%, more preferably 96%, more preferably 99%, more preferably 100% identical with a sequence complementary to TNC mRNA in the 472-492 nucleotide region of Seq ID No: 25, and/or comprises a molecule at least 85%, more preferably 90%, more preferably 96%, more preferably 99%, more preferably 100% identical with Seq ID No: 7; most preferably is a molecule with the sequence of Seq ID No: 7; (iv) is at least 85%, more preferably 96%, more preferably 99%, more preferably 100% identical with the complementary sequence of the TNC mRNA in the 495-515 nucleotide region of Seq ID No: 25, and/or comprises a molecule at least 85%, more preferably 90%, more preferably 96%, more preferably 99%, more preferably 100% identical with Seq ID No: 9; most preferably is a molecule with the sequence of Seq ID No: 9; (v) is at least 85%, more preferably 96%, more preferably 99%, more preferably 100% identical with a sequence complementary to TNC mRNA in the 538-558 nucleotide region of Seq ID No: 25, and/or comprises a molecule at least 85%, more preferably 90%, more preferably 96%, more preferably 99%, more preferably 100% identical with Seq ID No: 11, most preferably is a molecule with the sequence of Seq ID No: 11; (vi) is at least 85%, more preferably 96%, more preferably 99%, more preferably 100% identical with the complementary sequence of the TNC mRNA in the 4625-4645 nucleotide region of Seq ID No: 25, and/or comprises a molecule at least 85%, more preferably 90%, more preferably 96%, more preferably 99%, more preferably 100% identical with Seq ID No: 13, most preferably is a molecule with the sequence of Seq ID No: 13.

Preferably, the siRNA molecule is in the form of a double-stranded RNA (dsRNA) molecule with or without from 2 to 4 nucleotide overhangs, wherein the dsRNA consisting of a single-stranded ssRNA of the effector molecule and an ssRNA of the passenger molecule and wherein the duplex region is at least 21 nucleotides. Preferably it is between 21 and 30 nucleotides, most preferably 21-27 nucleotides.

Preferably, the siRNA molecule is a siRNA molecule selected from (i) MB-R-019 being a duplex of the effector sequence of Seq ID No: 3 with the passenger sequence of Seq ID No: 4; (ii) MB-R-047 being a duplex of the effector sequence of Seq ID No: 5 with the passenger sequence of Seq ID No: 6; (iii) MB-R-068 being a duplex of the effector sequence of Seq ID No: 7 with the passenger sequence of Seq ID No: 8; (iv) MB-R-091 being a duplex of effector sequence of Seq ID No: 9 with passenger sequence of Seq ID No: 10; (v) MB-R-134 being a duplex of effector sequence of Seq ID No: 11 with passenger sequence of Seq ID No: 12; (vi) siRNA-TNC being a duplex of effector sequence of Seq ID No: 13 with passenger sequence of Seq ID No: 14.

Preferably, the siRNA molecule comprises at least one chemically modified nucleotide and/or at least one modification selected from 2′-O-Me modification, PTO-type binding, 2′-Fluoro RNA modification, 5′-E vinylphosphonate 2′-methoxyuridine modification, modification with cholesterol, modification by deoxynucleotide attachment, preferably comprises attached deoxynucleotides [dTdT] at the 3′ end of the effector strand.

The invention also relates to a lipid nanoparticle LNP with siRNA, which comprises at least one siRNA molecule according to the invention.

Preferably, the LNP is a cationic lipid complex or an ionizable (neutral) lipid complex. Examples of applicable LNPs include LNP1-3 as defined herein.

Preferably, the LNP is an LNP with siRNA obtained by the microfluidic mixing method.

In the LNP variant, the cationic (LNP1) lipid complex is composed of a mixture of lipids: DSPC (1,2-distearoyl-sn-glycero-3-phosphocholine):DOTAP (N-[1-(2,3-dioleoyloxy)propyl]-N,N,N-trimethyl-ammonium chloride):cholesterol:DSPE-PEG2000-amine (1,2-distearoyl-sn-glycero-3-phosphoethanolamina-N-[amino(polyethylene glycol)-2000)] or DSPE-PEG2000 (1,2-distearoyl-sn-glycero-3-phosphoethanolamina-N(polyethylene glycol)-2000) or DMG-PEG2000 (1,2-dimyristoyl-rac-glycero-3-methoxypolyethylene glycol-2000) combined in a molar ratio of 10:40:48:2±10% of each lipid.

In the LNP variant, the ionizable (LNP2) lipid complex is composed of a mixture of lipids: DSPC (1,2-distearoyl-sn-glycero-3-phosphocholine):DLin-MC3 (4-(dimethylamino)-butanoate (10Z,13Z)-1-(9Z,12Z)-9,12-octadecadien-1-yl-10,13-nonadecadien-1-yl):cholesterol:DSPE-PEG2000-amine (1,2-distearoyl-sn-glycero-3-phosphoethanolamina-N-[amino(polyethylene glycol)-2000)] or DSPE-PEG2000 (1,2-distearoyl-sn-glycero-3-phosphoethanolamina-N(polyethylene glycol)-2000 or DMG-PEG2000 (1,2-dimyristoyl-rac-glycero-3-methoxypolyethylene glycol-2000) combined in a molar ratio of 10:40:48:2±10% of each lipid.

In the LNP variant, the ionizable (LNP3) lipid complex is composed of a mixture of lipids: DSPC (1,2-distearoyl-sn-glycero-3-phosphocholine):DLin-KC2-DMA (2,2-dilinoleyl-4-dimethylaminoethyl-[1,3]-dioxolane):cholesterol:DSPE-PEG2000-amine (1,2-distearoyl-sn-glycero-3-phosphoethanolamina-N-[amino(polyethylene glycol)-2000)] or DSPE-PEG2000 (1,2-distearoyl-sn-glycero-3-phosphoethanolamina-N(polyethylene glycol)-2000 or DMG-PEG2000 (1,2-dimyristoyl-rac-glycero-3-methoxypolyethylene glycol-2000) combined in a molar ratio of 10:50:38.5:1.5±10% of each lipid.

In the LNP variant, the lipid nanoparticle complex is an LNP1 complex composed of a mixture of DSPC at a concentration of 20±2 mg/mL, DOTAP at a concentration of 20±2 mg/mL, cholesterol at a concentration of 20±2 mg/mL, DSPE-PEG2000 or DMG-PEG2000 at a concentration of 20±2 mg/mL; wherein preferably the LNP1 nanoparticles with siRNA have a diameter of less than 120 nm and a polydispersity index of <0.1, more preferably the LNP1 nanoparticles with siRNA of less than 100 nm in diameter.

In the LNP variant, the lipid nanoparticle complex is an LNP2 complex composed of a mixture of DSPC at a concentration of 20±2 mg/mL, DLin-MC3-DMA at a concentration of 20±2 mg/mL, cholesterol at a concentration of 20±2 mg/mL, DSPE-PEG2000 or DMG-PEG2000 at a concentration of 20±2 mg/mL; wherein preferably the LNP2 nanoparticles with siRNA have a diameter of less than 120 nm and a polydispersity index of <0.1, more preferably the LNP2 nanoparticles with siRNA of less than 100 nm in diameter.

In the LNP variant, the lipid nanoparticle complex is an LNP3 complex composed of a mixture of DSPC at a concentration of 20±2 mg/mL, DLin-KC2-DMA at a concentration of 20±2 mg/mL, cholesterol at a concentration of 20±2 mg/mL, DSPE-PEG2000-amine or DSPE-PEG2000 or DMG-PEG2000 at a concentration of 20±2 mg/mL; wherein preferably the LNP3 nanoparticles with siRNA have a diameter of less than 120 nm and a polydispersity index of <0.1, more preferably the LNP3 nanoparticles with siRNA of less than 100 nm in diameter.

In the LNP1 or LNP2 or LNP3 variant, the lipid nanoparticle with siRNA is obtained by the microfluidic mixing method.

The invention also relates to a pharmaceutical composition comprising at least one siRNA according to the invention and/or at least one lipid nanoparticle LNP and a pharmaceutically acceptable carrier, vehicle or excipient.

The invention also relates to a pharmaceutical composition with anti-cancer properties comprising at least one siRNA according to the invention and/or at least one lipid nanoparticle LNP with a siRNA according to the invention and a pharmaceutically acceptable carrier, vehicle or excipient for use as a drug for the treatment and/or prevention of the development of cancer characterized by increased TNC expression in a human, by inhibiting TNC expression.

The pharmaceutical composition for use as a drug for the treatment and/or prevention of the development of cancer characterized by increased TNC expression in a mans is preferably used against cancer with increased TNC expression selected from glioma, breast cancer, ovarian cancer, and pancreatic cancer.

The invention also relates to a pharmaceutical composition with anticancer properties comprising a siRNA molecule for use in the therapy and/or prevention of glioma by inhibiting the expression of human TNC, the pharmaceutical composition comprising at least one siRNA molecule located in a lipid nanoparticle LNP complex and selected from the group consisting of: MB-R-019 being a duplex of the effector sequence of Seq ID No: 3 with the passenger sequence of Seq ID No: 4, MB-R-047 being a duplex of the effector sequence of Seq ID No: 5 with the passenger sequence of Seq ID No: 6, MB-R-091 being a duplex of the effector sequence of Seq ID No: 9 with the passenger sequence of Seq ID No: 10, siRNA-TNC being a duplex of the effector sequence of Seq ID No: 13 with the passenger sequence of Seq ID No: 14, or any mixture thereof; and a pharmaceutically acceptable carrier, vehicle or excipient.

In a variant of the pharmaceutical composition for use in the therapy and/or prevention of the development of a glioma, the lipid nanoparticle LNP complex is a cationic LNP1 lipid nanoparticle complex, composed of a lipid mixture: DSPC (1,2-distearoyl-sn-glycero-3-phosphocholine):DOTAP (N-[1-(2,3-dioleoyloxy)propyl]-N,N,N-trimethylammonium chloride):cholesterol:DSPE-PEG2000-amine (1,2-distearoyl-sn-glycero-3-phosphoethanolamine-N-[amino(polyethylene glycol)-2000)] or DSPE-PEG2000 (1,2-distearoyl-sn-glycero-3-phosphoethanolamine-N-(polyethylene glycol)-2000) or DMG-PEG2000 (1,2-dimyristoyl-rac-glycero-3-methoxypolyethylene glycol-2000), combined in a molar ratio of 10:40:48:2±10% of each lipid, more preferably the cationic lipid nanoparticle LNP complex is LNP1 composed of a mixture of: DSPC at a concentration of 20±2 mg/mL, DOTAP at a concentration of 20±2 mg/mL, cholesterol at a concentration of 20±2 mg/mL, DSPE-PEG2000-amine or DSPE-PEG2000 or DMG-PEG2000 at a concentration of 20±2 mg/mL; wherein preferably the LNP1 nanoparticles with siRNA have a diameter of less than 120 nm and a polydispersity index of <0.1, more preferably the LNP1 nanoparticles with siRNA of less than 100 nm in diameter.

In a variant of the pharmaceutical composition for use in the therapy and/or prevention of the development of glioma, LNP nanoparticles with siRNA were obtained by the microfluidic mixing method.

In a preferable pharmaceutical composition for use in the therapy and/or prevention of the development of glioma, the siRNA comprises at least one modification selected from 2′-O-Me modification, PTO-type binding, 2′-Fluoro RNA modification, 5′-E vinylphosphonate 2′-methoxyuridine modification, modification with cholesterol, modification by deoxynucleotide attachment, preferably comprising attached deoxynucleotides [dTdT] at the 3′ end of the effector strand.

The pharmaceutical composition for use in the therapy and/or prevention of the development of a glioma preferably comprises a mixture of at least two, more preferably three, more preferably four random selected siRNAs from MB-R-019, MB-R-047, MB-R-091, siRNA-TNC in a ratio of x:x:x:x or in a ratio of x:x:x or in a ratio of x:x, wherein x is in the range of 1 to 10.

The pharmaceutical composition for use in the therapy and/or prevention of the development of a glioma preferably comprises a mixture of at least two types of LNP nanoparticles with siRNA in a molar ratio of x:x, more preferably three types of LNP nanoparticles with siRNA in a molar ratio of x:x:x, more preferably four types of LNP nanoparticles with siRNA in a molar ratio of x:x:x:x, selected from MB-R-019, MB-R-047, MB-R-091, siRNA-TNC, wherein each type of LNP nanoparticles comprising a different siRNA and wherein x is in the range of 1 to 10.

The invention also relates to a pharmaceutical composition with anticancer properties comprising an siRNA molecule for use in the therapy and/or prevention of the development of a breast cancer by inhibiting the expression of human TNC, the pharmaceutical composition comprising at least one siRNA molecule located in a lipid nanoparticle LNP complex and selected from the group consisting of: MB-R-019 being a duplex of the effector sequence of Seq ID No: 3 with the passenger sequence of Seq ID No: 4, MB-R-047 being a duplex of the effector sequence of Seq ID No: 5 with the passenger sequence of Seq ID No: 6, MB-R-068 being a duplex of the effector sequence of Seq ID No: 7 with the passenger sequence of Seq ID No: 8, MB-R-091 being a duplex of the effector sequence of Seq ID No: 9 with the passenger sequence of Seq ID No: 10, MB-R-134 being a duplex of the effector sequence of Seq ID No: 11 with the passenger sequence of Seq ID No: 12, siRNA-TNC being a duplex of the effector sequence of Seq ID No: 13 with the passenger sequence of Seq ID No: 14; or any mixture thereof; and alternatively a pharmaceutically acceptable carrier, vehicle or excipient.

In a variant of the pharmaceutical composition for use in the therapy and/or prevention of the development of a breast cancer, the lipid nanoparticle LNP complex is a cationic LNP1 lipid nanoparticle complex, composed of a lipid mixture: DSPC (1,2-distearoyl-sn-glycero-3-phosphocholine):DOTAP (N-[1-(2,3-dioleoyloxy)propyl]-N,N,N-trimethylammonium chloride):cholesterol:DSPE-PEG2000-amine (1,2-distearoyl-sn-glycero-3-phosphoethanolamine-N-[amino(polyethylene glycol)-2000), combined in a weight ratio of 10:40:48:2±10% of each lipid, more preferably the cationic lipid nanoparticle LNP complex is LNP1 composed of a mixture of DSPC at a concentration of 20±2 mg/mL, DOTAP at a concentration of 20±2 mg/mL, cholesterol at a concentration of 20±2 mg/mL, DSPE-PEG2000 at a concentration of 20±2 mg/mL; wherein preferably the LNP1 nanoparticles with siRNA have a diameter of less than 120 nm and a polydispersity index of <0.1, more preferably the LNP1 nanoparticles with siRNA of less than 100 nm in diameter.

In a variant of the pharmaceutical composition for use in the therapy and/or prevention of the development of breast cancer, LNP nanoparticles with siRNA were obtained by the microfluidic mixing method.

In a variant of the pharmaceutical composition for use in the therapy and/or prevention of cancers that is the subject of this document, the lipid nanoparticle LNP complex may also be lipid nanoparticles LNP2 or LNP3 complex with the qualitative and quantitative compositions listed herein.

The pharmaceutical composition for use in the therapy and/or prevention of the development of breast cancer preferably comprises at least one modification selected from 2′-O-Me modification, PTO-type binding, 2′-Fluoro RNA modification, 5′-E vinylphosphonate 2′-methoxyuridine modification, modification with cholesterol, modification by deoxynucleotide attachment, preferably comprises attached deoxynucleotides [dTdT] at the 3′ end of the effector strand.

The pharmaceutical composition for use in the therapy and/or prevention of the development of breast cancer preferably comprises a mixture of at least two, more preferably three, more preferably four, more preferably five, more preferably six random selected siRNAs from MB-R-019, MB-R-047, MB-R-068, MB-R-091, MB-R-134, siRNA-TNC in the ratio of x:x:x:x:x:x, in the ratio of x:x:x:x:x, in the ratio of x:x:x:x or in the ratio of x:x:x or in the ratio of x:x, wherein x is in the range of 1 to 10.

The pharmaceutical composition for use in the therapy and/or prevention of the development of breast cancer preferably comprises a mixture of at least two types of LNP nanoparticles with siRNA in a molar ratio of x:x, more preferably three types of LNP nanoparticles with siRNA in a molar ratio of x:x:x, more preferably four types of LNP nanoparticles with siRNA in a molar ratio of x:x:x:x, more preferably five LNPs with siRNA in a molar ratio of x:x:x:x:x, more preferably six LNPs with siRNA in a molar ratio of x:x:x:x:x:x, selected from MB-R-019, MB-R-047, MB-R-068, MB-R-091, MB-R-134, siRNA-TNC, wherein each type of LNP nanoparticles comprising a different siRNA and wherein x is in the range of 1 to 10.

The invention also relates to a pharmaceutical composition with anticancer properties comprising an siRNA molecule for use in the therapy and/or prevention of the development of an ovarian cancer by inhibiting the expression of human TNC, the pharmaceutical composition comprising at least one siRNA molecule located in a lipid nanoparticle LNP complex and selected from the group consisting of: MB-R-019 being a duplex of the effector sequence of Seq ID No: 3 with the passenger sequence of Seq ID No: 4, MB-R-068 being a duplex of the effector sequence of Seq ID No: 7 with the passenger sequence of Seq ID No: 8, MB-R-091 being a duplex of the effector sequence of Seq ID No: 9 with the passenger sequence of Seq ID No: 10, MB-R-134 being a duplex of the effector sequence of Seq ID No: 11 with the passenger sequence of Seq ID No: 12, siRNA-TNC being a duplex of the effector sequence of Seq ID No: 13 with the passenger sequence of Seq ID No: 14, or any mixture thereof; and alternatively a pharmaceutically acceptable carrier, vehicle or excipient.

In a variant of the pharmaceutical composition for use in the therapy and/or prevention of the development of an ovarian cancer, the lipid nanoparticle LNP complex is a cationic LNP1 lipid nanoparticle complex, composed of a lipid mixture: DSPC (1,2-distearoyl-sn-glycero-3-phosphocholine):DOTAP (N-[1-(2,3-dioleoyloxy)propyl]-N,N,N-trimethylammonium chloride):cholesterol:DSPE-PEG2000-amine (1,2-distearoyl-sn-glycero-3-phosphoethanolamine-N-[amino(polyethylene glycol)-2000), combined in a weight ratio of 10:40:48:2±10% of each lipid, more preferably the cationic lipid nanoparticle LNP complex is LNP1 composed of a mixture of DSPC at a concentration of 20±2 mg/mL, DOTAP at a concentration of 20±2 mg/mL, cholesterol at a concentration of 20±2 mg/mL, DSPE-PEG2000-amine at a concentration of 20±2 mg/mL, more preferably the LNP1 nanoparticles with siRNA have a diameter of less than 120 nm and a polydispersity index of <0.1, more preferably the LNP1 nanoparticles with siRNA of less than 100 nm in diameter.

In a variant of the pharmaceutical composition for use in the therapy and/or prevention of the development of ovarian cancer, LNP nanoparticles with siRNA were obtained by the microfluidic mixing method.

The pharmaceutical composition for use in the therapy and/or prevention of the development of an ovarian cancer preferably comprises at least one modification selected from 2′-O-Me modification, PTO-type binding, 2′-Fluoro RNA modification, 5′-E vinylphosphonate 2′-methoxyuridine modification, modification with cholesterol, modification by deoxynucleotide attachment, preferably comprising attached deoxynucleotides [dTdT] at the 3′ end of the effector strand.

The pharmaceutical composition for use in the therapy and/or prevention of the development of ovarian cancer preferably comprises a mixture of at least two, more preferably three, more preferably four, more preferably five random selected siRNAs from MB-R-019, MB-R-068, MB-R-091, MB-R-134, siRNA-TNC in a ratio of x:x:x:x:x, in a ratio of x:x:x:x or in a ratio of x:x:x or in a ratio of x:x, wherein x is in the range of 1 to 10.

The pharmaceutical composition for use in the therapy and/or prevention of the development of ovarian cancer preferably comprises a mixture of at least two types of LNP nanoparticles with siRNA in a molar ratio of x:x, more preferably three types of LNP nanoparticles with siRNA in a molar ratio of x:x:x, more preferably four types of LNP nanoparticles with siRNA in a molar ratio of x:x:x:x, more preferably five LNPs with siRNA in a molar ratio of x:x:x:x:x, selected from MB-R-019, MB-R-068, MB-R-091, MB-R-134, siRNA-TNC, wherein each type of LNP nanoparticles comprises a different siRNA and wherein x is in the range from 1 to 10.

The invention also relates to a pharmaceutical composition with anticancer properties comprising an siRNA molecule for use in the therapy and/or prevention of the development of pancreatic cancer by inhibiting the expression of human TNC, the pharmaceutical composition comprising at least one siRNA molecule located in a lipid nanoparticle LNP complex and selected from the group consisting of: MB-R-019 being a duplex of the effector sequence of Seq ID No: 3 with the passenger sequence of Seq ID No: 4, MB-R-047 being a duplex of the effector sequence of Seq ID No: 5 with the passenger sequence of Seq ID No: 6, MB-R-068 being a duplex of the effector sequence of Seq ID No: 7 with the passenger sequence of Seq ID No: 8, MB-R-091 being a duplex of the effector sequence of Seq ID No: 9 with the passenger sequence of Seq ID No: 10, MB-R-134 being a duplex of the effector sequence of Seq ID No: 11 with the passenger sequence of Seq ID No: 12, siRNA-TNC being a duplex of the effector sequence of Seq ID No: 13 with the passenger sequence of Seq ID No: 14, or any mixture thereof; and alternatively a pharmaceutically acceptable carrier, vehicle or excipient.

In a variant of the pharmaceutical composition for use in the therapy and/or prevention of the development of pancreatic cancer, the lipid nanoparticle LNP complex is a cationic LNP1 lipid nanoparticle complex composed of a lipid mixture: DSPC (1,2-distearoyl-sn-glycero-3-phosphocholine):DOTAP (N-[1-(2,3-dioleoyloxy)propyl]-N,N,N-trimethylammonium chloride):cholesterol:DSPE-PEG2000-amine (1,2-distearoyl-sn-glycero-3-phosphoethanolamine-N-[amino(polyethylene glycol)-2000), combined in a weight ratio of 10:40:48:2±10% of each lipid, more preferably the cationic lipid nanoparticle LNP complex is LNP1 composed of a mixture of DSPC at a concentration of 20±2 mg/mL, DOTAP at a concentration of 20±2 mg/mL, cholesterol at a concentration of 20±2 mg/mL, DSPE-PEG2000-amine at a concentration of 20±2 mg/mL, wherein more preferably the nanoparticles LNP1 with siRNA have a diameter of less than 120 nm and a polydispersity index of <0.1, more preferably the nanoparticles LNP1 with siRNA have a diameter of less than 100 nm.

In a variant of the pharmaceutical composition for use in the therapy and/or prevention of the development of pancreatic cancer, LNP nanoparticles with siRNA were obtained by microfluidic mixing method.

The pharmaceutical composition for use in the therapy and/or prevention of the development of pancreatic cancer preferably comprises at least one modification selected from 2′-O-Me modification, PTO-type binding, 2′-Fluoro RNA modification, 5′-E vinylphosphonate 2′-methoxyuridine modification, modification with cholesterol, modification by deoxynucleotide attachment, preferably comprising attached deoxynucleotides [dTdT] at the 3′ end of the effector strand.

The pharmaceutical composition for use in the therapy and/or prevention of the development of pancreatic cancer preferably comprises a mixture of at least two, more preferably three, more preferably four, more preferably five, more preferably six random selected siRNAs from MB-R-019, MB-R-047, MB-R-068, MB-R-091, MB-R-134, siRNA-TNC in the ratio of x:x:x:x:x:x, in the ratio of x:x:x:x:x, in the ratio of x:x:x:x or in the ratio of x:x:x or in the ratio of x:x, wherein x is in the range of 1 to 10.

The pharmaceutical composition for use in the therapy and/or prevention of the development of pancreatic cancer preferably comprises a mixture of at least two types of LNP nanoparticles with siRNA in a molar ratio of x:x, more preferably three types of LNP nanoparticles with siRNA in a molar ratio of x:x:x, more preferably four types of LNP nanoparticles with siRNA in a molar ratio of x:x:x:x, more preferably five LNPs with siRNA in a molar ratio of x:x:x:x:x, more preferably six LNPs with siRNA in a molar ratio of x:x:x:x:x:x, selected from MB-R-019, MB-R-047, MB-R-068, MB-R-091, MB-R-134, siRNA-TNC, wherein each type of LNP nanoparticles comprising a different siRNA and wherein x is in the range of 1 to 10.

It was unexpectedly found that only selected siRNAs among all the molecules developed and tested could be used as interfering RNA to block TNC expression in cancer cells, thus inhibiting the development of malignant cancers: brain including glioma, breast, ovary, pancreas, in which an increase in TNC levels are observed. By examining which specific siRNAs effectively silence TNC expression, it was possible to determine within the TNC gene the sites with the highest silencing potential, particularly within the ATN region within TNC. Such developed siRNAs according to the invention, also in the form of lipid LNP complexes with siRNA-TNC, MB-R-019, MB-R-047, MB-R-068, MB-R-091, MB-R-134 are administered to inhibit the development of the above-mentioned cancers, their treatment and will improve the quality of life of patients and extend their lifespan.

It also turned out that the siRNA molecules according to the invention, compared to siRNA-ATN, are less toxic and have no immunogenic effect.

It was unexpectedly found that lipid LNP complexes with selected siRNAs according to the invention (siRNA-TNC, MB-R-019, MB-R-047, MB-R-068, MB-R-091, MB-R-134) or mixtures thereof, or mixtures of such lipid complexes, allow to obtain a stable formulation improving the delivery of selected siRNA, increasing the stability of the siRNA molecules packed therein, increasing the half-life of the RNAs and the efficiency of their delivery and action in the target cancer cell, which allowed to obtain selective inhibition of TNC expression in cancer cells. At the same time, the LNP carriers used proved to be not only extremely effective but also very safe, as demonstrated in vivo in toxicity tests.

In addition, LNP complexes with selected siRNAs (siRNA-TNC, MB-R-019, MB-R-047, MB-R-068, MB-R-091, MB-R-134) or their mixtures are synthesized using the microfluidic mixing method, which, in comparison with known state-of-the-art methods of lipid film hydration or ethanol dilution, allows to obtain a homogeneous, small diameter fraction of nanoparticles, and thus this eliminates the need for their further processing, i.e. sonication, extrusion, sorting and specialized purification, and thus there is no damage to siRNA during the formation of lipid complexes comprising it, which affects the efficacy of therapy with lipid complexes obtained according to the invention.

Cationic lipid LNP complexes with selected siRNA according to the invention, or mixtures thereof, can be delivered directly into the postsurgical locus after removed tumour, thus ensuring safe and local action, significantly limiting the potential occurrence of side effects. Topical administration of the therapeutic agent enhances its efficacy within cancer and lesional tissue and contributes to limiting the infiltration processes that often accompany cancers, in particular brain tumours that cannot be completely removed surgically.

At the same time, chemical modifications of siRNA molecules selected for efficiency, selected from 2′-O-Me modification, PTO-type binding, 2′-Fluoro RNA modification, 5′-E vinylphosphonate 2′-methoxyuridine modification, modification with cholesterol, modification by addition of deoxynucleotides [dTdT](e.g. at the 3′ end of the effector strand) that they comprise in relation to the basic sequence of the siRNA molecule according to the invention further improve their stability, efficiency and specificity, and thus influence better efficiency and silencing of TNC expression using them.

The selected siRNA molecules with the most preferable properties, i.e. the highest efficiency in silencing TNC gene expression, are at the same time characterized by the lowest induction of expression of early immune response genes (RIG1 and OAS1), making them safe as therapeutic agents.

It was unexpectedly found that also the mixture of siRNA molecules according to the invention: siRNA-TNC, MB-R-019, MB-R-047, MB-R-068, MB-R-091, MB-R-134, in equal or varying ratios, at least two, preferably three, preferably four, preferably five, preferably six siRNAs according to the invention, preferably in a ratio between 1:1 and 1:10 of each component depending on the specific composition, especially administered as nanoparticles LNP of lipid complexes, shows a positive effect of expression silencing. A synergistic effect has been demonstrated in the cooperation of siRNA molecules according to the invention in inhibiting TNC expression in cancer cells of glioma, breast cancer, ovarian cancer, and pancreatic cancer. Such siRNA mixture compositions according to the invention can be administered as a mixture of two or three or four or five or six different types of siRNA molecules, particularly preferably in the form of lipid LNP complexes comprising siRNAs embedded in the lipid LNP complexes. The mixture of siRNA molecules according to the invention may therefore comprise all 6: siRNA-TNC:MB-R-019:MB-R-047:MB-R-068:MB-R-091:MB-R-134 in the ratio of x:x:x:x:x:x, any 5 of 6 in the ratio of x:x:x:x:x, any 4 of 6 in the ratio of x:x:x:x; any 3 of 6 in the ratio of x:x:x; any 2 of 6 in the ratio of x:x, wherein x is in the range of 1 to 10. In particular, preferably mixtures of two or more siRNA molecules according to the invention are equimolar mixtures.

Unless otherwise defined, all technical and scientific terms used herein have the meanings commonly understood by those skilled in the art to which this invention belongs. The following terms, and the abbreviations used, have the meanings assigned to them below unless otherwise defined. GBM—glioblastoma; OV—ovarian cancer; BRCA—breast cancer; PAAD—pancreatic adenocarcinoma; ATN-RNA—double-stranded RNA (dsRNA) molecule with 163 nt in length—disclosed in the state of the art (Seq ID 1 and 2 hybrid), corresponding at position 405-567 to a TNC fragment (human TNC transcript sequence disclosed in NCBI database www.ncbi.nlm.nih.gov under the number NM_002160.4, and presented as Seq ID No: 25); siRNA-ATN-siRNA molecules within the ATN-RNA fragment, various siRNA-ATNs, including the subject of the application are named according to the formula: MB-R-three digit number, corresponding to their position relative to the ATN-RNA molecule; siRNA-TNC-siRNA molecule within a non-ATN-RNA fragment of the TNC sequence; LNP-lipid nanoparticles (both cationic and ionizable); LNP1—cationic LNPs (LNP1 and the specified RNA are lipid nanoparticles with the selected RNA according to the invention); LNP2—ionizable (neutral) LNPs based on DLin-MC3-DMA; LNP3-ionizable (neutral) LNPs based on DLin-KC2-DMA; LNP1-ATN-RNA—a complex of cationic lipid and ATN-RNA obtained by microfluidic mixing method; LNP2-ATN-RNA—a complex of ionizable lipid and ATN-RNA obtained by microfluidic mixing method; LPN1-siRNA-TNC—a complex of cationic lipids and siRNAs of the non-ATN sequence obtained by microfluidic mixing method; LNP1-siRNA-ATN—a complex of cationic lipids and siRNAs within the ATN sequence, which includes LNP1-MB-R—a three digit number which denotes the complex of cationic lipid and specific siRNA-ATN (i.e. LNP1-MB-R-019, LNP1-MB-R-047, LNP1-MB-R-068, LNP1-MB-R-091, LNP1-MB-R-134); LNP3-siRNA-TNC—a complex of ionizable lipids and siRNAs of the non-ATN sequence obtained by microfluidic mixing method; LNP3-siRNA-ATN—a complex of ionizable lipids and specific siRNA-ATNs (i.e. LNP3-siRNA-TNC, LNP3-MB-R-019, LNP3-MB-R-047, LNP3-MB-R-068, LNP3-MB-R-091, LNP3-MB-R-134); L2000-Lipofectamine™ 2000 (Invitrogen), OAS1-2′-5′-oligoadenylate synthetase; RIG1—retinoic acid-inducible gene 1; TNC—tenascin-C.

### BRIEF DESCRIPTION OF DRAWINGS

Embodiments of the invention are presented in the figures, in which:

FIG. 1 Shows a box plot demonstrating the comparison of TNC mRNA level in cancer cells of glioma (GBM), ovarian cancer (OV), breast cancer (BRCA), and pancreatic cancer (PAAD) relative to healthy tissue. The analysis was performed using data from The Cancer Genome Atlas (TCGA) database for various cancers. GBM, OV, BRCA, and PAAD cancers (cancer tissue—left side, healthy tissue—right side of this plot) exhibited elevated TNC expression compared to tissues from healthy humans.

FIG. 2 Shows the relative TNC mRNA level determined by RT-qPCR analysis in the tested cell lines (A) U251-MG glioma, (B) MDA-MB-231 breast cancer, (C) OVCAR-3 ovarian cancer, (D) PANC-1 pancreatic cancer after transfection with the indicated siRNA-ATN, siRNA-TNC or ATN-RNA and L2000 carrier alone. Control: C—carrier alone; siRNA-ATNs tested with carrier: MB-R-019, MB-R-047, MB-R-068, MB-R-091, MB-R-134 (selected siRNAs from within the ATN-RNA fragment); siRNA-TNC with carrier [less=better].

FIG. 3 Shows cell viability analysis after treatment with therapeutic complexes with ATN-RNA, siRNA-TNC or siRNA-ATN against (A) U251-MG glioma, (B) MDA-MB-231 breast cancer, (C) OVCAR-3 ovarian cancer, (D) PANC-1 pancreatic cancer cell lines using L2000 for transfection. Control: C0—untreated cells; C—carrier alone, tested siRNA-ATN with carrier: MB-R-019, MB-R-047, MB-R-068, MB-R-091, MB-R-134; siRNA-TNC with carrier (siRNA of the non-ATN-RNA) [more=better].

FIG. 4 Shows the results of OAS1 and RIG1 expression levels determined by RT-qPCR analysis in (A) U251-MG glioma, (B) MDA-MB-231 breast cancer, (C) OVCAR-3 ovarian cancer, (D) PANC-1 pancreatic cancer cell lines after transfection with the indicated siRNA-ATN, siRNA-TNC or ATN-RNA and L2000 carrier alone. Control: C L2000—carrier alone; siRNA-ATN molecules tested with carrier: MB-R-019, MB-R-047, MB-R-068, MB-R-091, MB-R-134; siRNA-TNC with carrier (siRNA of the non-ATN-RNA) [less=better].

FIG. 5 Shows relative TNC mRNA level determined by RT-qPCR analysis in (A) U251-MG glioma, (B) MDA-MB-231 breast cancer, (C) OVCAR-3 ovarian cancer and (D) PANC1 pancreatic cancer lines after treatment with mixtures of siRNA: MB-R-019, MB-R-047, MB-R-068, MB-R-091 and MB-R-134 and siRNA-TNC in complexes with L2000 carrier. Controls: Cscr—cells treated with L2000 and a bio-logically inactive siRNA molecule (scr). Bar captions indicate specific RNA/RNA mixture [less=better].

FIG. 6 Shows cell viability analysis of (A) U251-MG glioma, (B) MDA-MB-231 breast cancer, (C) OVCAR-3 ovarian cancer, (D) PANC-1 pancreatic cancer after ATN-RNA treatment in the presence of different carriers. Control: C0—untreated cells. ATN-RNA-L2000 carrier with ATN-RNA, LNP1-ATN-RNA—cationic lipids with ATN-RNA. (A) U251-MG glioma, (B) MDA-MB-231 breast cancer, (C) OVCAR-3 ovarian cancer (D) MRC-5 non-cancer cells [more=better].

FIG. 7 Shows an examination of TNC mRNA level in U118-MG and U251-MG human glioma cell lines after ATN-RNA transfection using two types of LNP carrier. LNP1-ATN-RNA-ATN-RNA in complex with cationic lipids, LNP2-ATN-RNA-ATN-RNA in complex with ionizable lipids, C LNP1-control-carrier alone, C LNP2-control-carrier alone [less=better].

FIG. 8 Shows an examination of TNC mRNA level after ATN-RNA treatment and various siRNAs in complex with LNPL. (A) U251-MG glioma line, (B) MDA-MB-231 breast cancer line, (C) OVCAR-3 ovarian cancer line, (D) PANC-1 pancreatic cancer line. Controls: C1 LNP1, C2 LNP1—treated with carrier alone in the amount corresponding to the amount needed to deliver the corresponding RNA concentration (0.7 μg/mL and 1.4 μg/mL, respectively). Bar captions indicate the complex of the LNP1 carrier+specific RNA (ATN-RNA; selected siRNA-ATN: MB-R-019, MB-R-091; siRNA-TNC), for each line tested at two concentrations (0.7 μg/mL and 1.4 μg/mL, respectively) [less=better].

FIG. 9 Shows relative TNC mRNA level determined by RT-qPCR analysis in (A) U251-MG glioma, (B) MDA-MB-231 breast cancer, (C) OVCAR-3 ovarian cancer and (D) PANC-1 pancreatic cancer lines after treatment with mixtures of LNP3 complexes with different siRNAs: MB-R-019, MB-R-091 and siRNA-TNC. Controls: C0—untreated cells, C LNP3—cells treated with LNP carrier. Bar captions indicate the complex of the LNP carrier with a specific siRNA or mixture of siRNAs [less=better].

FIG. 10 Shows cell viability analysis of (A) U251-MG glioma, (B) MDA-MB-231 breast cancer, (C) OVCAR-3 ovarian cancer, (D) PANC-1 pancreatic cancer after ATN-RNA treatment and various siRNAs in complex with LNPL. Control: C0—untreated cells, C1 LNP1, C2 LNP1—treated with carrier alone in the amount corresponding to the amount needed to deliver the corresponding RNA concentration (0.7 μg/mL and 1.4 μg/mL, respectively). Bar captions indicate the complex of the LNP1 carrier+specific RNA (ATN-RNA; selected siRNA-ATN: MB-R-019, MB-R-091; siRNA-TNC), for each line tested at two concentrations (0.7 μg/mL and 1.4 μg/mL, respectively) [more=better].

FIG. 11 Shows cell viability analysis of (A) U251-MG glioma, (B) MDA-MB-231 breast cancer, (C) OVCAR-3 ovarian cancer, (D) PANC-1 pancreatic cancer after treatment with different siRNAs in complex with LNP3. Control: C0—untreated cells, C1 LNP3, C2 LNP3—treated with carrier alone in the amount corresponding to the amount needed to deliver the corresponding RNA concentration (0.7 μg/mL and 1.4 μg/mL, respectively). Bar captions indicate the complex of the LNP3 carrier+selected siRNA-ATN: MB-R-019, MB-R-091; siRNA-TNC, for each line tested at two concentrations (0.7 μg/mL and 1.4 μg/mL, respectively) [more=better].

FIG. 12 Shows an examination of the OAS1 and RIG1 expression levels after transfection with different RNAs in complex with LNP1. (A) U251-MG glioma line, (B) MDA-MB-231 breast cancer line, (C) OVCAR-3 ovarian cancer line, (D) PANC-1 pancreatic cancer line. Control: LNP1 carrier alone. Bar captions indicate the complex of LNP1 carrier+specific RNA (ATN-RNA; selected siRNA-ATN: MB-R-019, MB-R-091; siRNA-TNC) [less=better].

FIG. 13 Shows the evaluation of the stability of (A) ATN-RNA and (B) siRNA-TNC as naked molecules (without carrier) and their lipid complexes in LNP1 in a human serum at 0-72 h.

FIG. 14 Shows the evaluation of the effect of selected chemical modifications on the stability of MB-R-019 in human serum at 0-240 min.

Cytowane w opisie publikacje oraz podane w nich odniesienia sa̧ w cal/ości niniejszym wl/a̧czone, jako referencje. Poniżize przykl/ady ilustruja̧wynalazek, nie ograniczaja̧c go w żaden sposób.

### DESCRIPTION OF EMBODIMENTS

The publications cited in the description and the references given therein are hereby incorporated in their entirety as references. The following examples illustrate the invention without limiting it in any way.

### EXAMPLES

### Materials and Methods

In the examples below, unless otherwise indicated, standard biochemical materials and methods were used or the manufacturers recommendations for specific materials and methods were followed. In the examples below, the following materials, cell lines, media and methods, procedures and tests were used repeatedly:

### (I) Cell Lines, Media

Table 1 below indicates the cell lines used, together with the culture media used for their culture

TABLE 1
List of cell lines used together with
the media used for their culture.

| | | | Culture |
|---|---|---|---|
| Name | Cell type | Source | medium used* |
| U-118 MG | glioma | ATCC | DMEM |
| U-251 MG | glioma | Sigma- | MEM |
| | | Aldrich | |
| MDA-MB- | triple-negative breast | ATCC | DMEM |
| 231 | cancer characterized by | | |
| | the absence of estrogen | | |
| | and progesterone | | |
| | receptors and overex- | | |
| | pression of HER-2 | | |
| OVCAR-3 | ovarian cancer | ATCC | RPMI 1640 |
| PANC-1 | pancreatic cancer | ATCC | DMEM |
| MRC-5 | non-cancer line derived | ATCC | MEM |
| | from normal fetal lung | | |
| | tissue | | |

*DMEM (Dulbecco's Modified Eagle's Medium) (Bio-West cat. no. L0101), MEM (Minimum Essential Medium) (Bio-West cat. no. L0430), RPMI 1640 (Bio-West cat. no. L0501), McCoy's 5A (Bio-West cat. no. L0210) supplemented with 10% calf serum (EURx cat. no. E5051-02), 1% stable glutamine (Bio-West cat. no. X0551), penicillin (100 μg/mL), streptomycin (100 mg/mL) (Bio-West cat. no. L0010) and for OVCAR-3 human insulin (10 μg/mL), unless otherwise indicated.

(II) Preparation of Lipid Nanoparticle Complexes of with Different RNAs

Lipid nanoparticles were obtained by microfluidic mixing method in a Nano Assemblr Benchtop apparatus (Precision Nanosystem).

Working solutions of DSPC (20 mg/mL), DOTAP (20 mg/mL), cholesterol (20 mg/mL) and DSPE-PEG 2000 (20 mg/mL) in ethanol, mixed in a molar ratio of 10:40:48:2 were used to prepare cationic lipids LNP1 and a ATN-RNA solution (100 μg) or specific siRNA (siRNA-ATN with selected sequences designated by the indicated numbers or siRNA-TNC) (100 μg) in 100 mM citrate buffer, pH 6.0.

Working solutions of DSPC (20 mg/mL), DLin-MC3-DMA (20 mg/mL), cholesterol (20 mg/mL) and DSPE-PEG 2000 (20 mg/mL) in ethanol, mixed in a molar ratio of 10:40:48:2 were used to prepare ionizable (neutral) lipids LNP2 and a ATN-RNA solution (100 μg) or specific siRNA (siRNA-ATN with selected sequences designated by the indicated numbers or siRNA-TNC) (100 μg) in 100 mM citrate buffer pH 6.0.

Working solutions of DSPC (20 mg/mL), DLin-KC2-DMA (20 mg/mL), cholesterol (20 mg/mL), DSPE-PEG2000-amine (20 mg/mL) or DSPE-PEG2000 (20 mg/mL) or DMG-PEG2000 (20 mg/mL) in ethanol, mixed in a ratio of 10:38.5:50:1.5 were used to prepare ionizable (neutral) lipids LNP3 and a solution of the specified siRNA (siRNA-ATN with selected sequences designated by the indicated numbers or siRNA-TNC) (100 μg) in 100 mM citrate buffer, pH 6.0.

A solution of the lipid components in ethanol and a solution of ATN-RNA or siRNA in citrate buffer were subjected to mixing at a volume ratio of 1:3, using a flow rate of 12 mL/min. The resulting lipid nanoparticle dispersion was dialyzed (lh, 4° C., 130 rpm) in phosphate-buffered saline (PBS) (0.137 mol/L sodium chloride, 0.0027 mol/L potassium chloride, 0.01 mol/L phosphate buffer) pH 7.2-7.6, using a 10 kDa MWCO regenerated cellulose dialysis membrane. To ensure the sterility of the product, the resulting suspensions of lipid nanoparticle complexes were filtered through a sterilising filter with a pore diameter of no more than 220 nm. The resulting dispersion was then transferred to vials and stored at 4-8° C.

In order to characterize the obtained lipids, their physicochemical properties were measured. The average size, zeta potential and polydispersity index of the lipids were determined using the DLS method (Malvern Zetasizer Nano ZS). Results were obtained in backscattering mode at 25° C. Each analysis consisted of three mea-surements, from which the mean size was calculated. Incorporation efficiency and the degree of binding were evaluated by applying both RNA-bound and RNA-unbound lipid complexes onto a 1.2% agarose gel. Densitometric analysis of the signals (Image J, Fiji) was then performed and the obtained values were compared.

Abbreviations of substances used mean: DSPC-1,2-distearoyl-sn-glycero-3-phosphocholine (CAS: 816-94-4); DOTAP-N-[1-(2,3-dioleoyloxy)propyl]-N,N,N-trimethylammonium chloride (CAS: 132172-61-3); DSPE-PEG2000-amine-1,2-distearoyl-sn-glycero-3-phosphoethanolamine-N-[amino(polyethylene glycol)-2000](CAS: 474922-26-4); DSPE-PEG2000-1,2-distearoyl-sn-glycero-3-phosphoethanolamine-N-(polyethylene glycol)-2000 (CAS: 474922-26-4); DMG-PEG2000-1,2-dimyristoyl-rac-glycero-3-methoxy-polyethylene glycol-2000 (CAS: 160743-62-4); MC-3 (DLin-MC3-DMA)-((6Z,9Z,28Z,31Z)-heptatriaconta-6,9,28,31-tetraen-19-yl-4-(dimethylamino) butanoate) (CAS: 1224606-06-7); KC2 (DLin-KC2-DMA)-2,2-dilinoleyl-4-dimethylaminoethyl-[1,3]-dioxolane (CAS: 1190197-97-7)

(III) Determination of the Stability of RNA Molecules Alone and in Complex with LNP1

To 500 ng of ATN-RNA or the specified siRNA and, for comparative studies, also an equimolar amount of the LNP1-ATN-RNA and LNP1-siRNA-ATN or LNP1-siRNA-TNC complex—produced as indicated in (II), 5 μL each of human serum (Sigma, cat. no. H4522) was added and the total was incubated at 37° C. for 0, 3 h, 6 h, 24 h, 48 h, 72 h, respectively. For the modified MB-R-019 molecules, incubation at 37° C. was carried out for 0, 5 min, 15 min, 30 min, 60 min, 120 min, 240 min, respectively. RNA degradation carried out by nucleases contained in the serum was stopped by the addition of 0.5 μL (20 U) of an RNAse inhibitor (Protector RNase Inhibitor, Roche). Samples were mixed with loading buffer and separated on a 1.2-2% agarose gel. The degree of RNA degradation, a measure of the stability of the complexes, was evaluated by densitometric signal intensity analysis (Image J, Fiji) for individual samples, unless otherwise indicated.

(IV) Cultivation of Cell Lines and their Transfection

Cells of a specific cell line were seeded in 12-well plates, maintaining a density of 50-100,000 cells/well, or in 96-well plates seeded at about 10,000 cells/well. The cells were cultured in their dedicated medium suitably supplemented according to (I) above. Cultures were conducted in incubators providing optimal growth conditions for the animal cells, i.e.: CO2 concentration 5%, temperature 37° C., humidity 95%.

Cells were transfected at about 70% confluence. The medium was exchanged for its non-supplemented counterpart enriched with the transfection mixture. The transfection mixture consisted of Lipofectamine™ 2000 (L2000) reagent (Invitrogen) alone or mixed according to the manufacturer's recommendations with the tested RNA (ATN-RNA or siRNA) or a specific lipid complex formed as in (II) with or without the tested RNA. For control samples to which transfection factor alone or empty LNP was added, the amounts needed to transfect the corresponding samples with RNA were used. After 48 h, cells were harvested and further analyses were performed on them (e.g. RNA isolation and RT-qPCR—see (VI)), unless otherwise indicated.

### (V) Evaluation of Cytotoxicity

Cell metabolic activity (cytotoxicity) was evaluated by a colorimetric assay with 3-(4,5-dimethyl-2-thiazolyl)-2,5-diphenyl-2H-tetrazolium bromide (MTT), unless otherwise indicated. The test is based on the ability of mitochondrial dehydrogenase to convert the orange, water-soluble tetrazolium salt to an insoluble dark blue formazan. The experiment was carried out in 96-well plates. Four wells were left blank on the plate to provide a reading background in later steps. Cells were seeded at about 10,000 per well and cultured for 2 h in supplemented medium under optimal growth conditions for the cell line as given in (II). The culture media was then exchanged for its non-supplemented counterpart and tested for cytotoxicity, carriers, lipid nanoparticles and their complexes with RNA were added in a final volume of 100 μL. Cytotoxicity assays were performed for two concentrations of RNA: 0.7 and 1.4 μg/mL, corresponding to amounts of 0.07 and 0.14 μg of RNA per single well. The amount of carrier (L2000) and RNA-free lipid nanoparticles (LNP1) used for the assay were chosen to correspond to their concentration in complexes with RNA. Incubation was carried out for 48 h in an incubator with 5% CO2.

A solution of MTT in non-supplemented culture medium with a final concentration of 0.5 mg/mL was prepared. Cells were washed with PBS solution. 100 μL each of MTT solution was applied to the wells and the plate was incubated 90 min in 5% CO2, 37° C. The MTT solution was then removed and the wells were flooded with 100 μL of DMSO to dissolve the formazan salt. The whole was shaken for 10 min. The lid was pulled off the plate and the absorbance was read at 590 nm (formazan) and 670 nm (MTT).

### (VI) RNA Isolation and RT-qPCR Analysis

Total RNA from cell lines was isolated using RNA Extracol reagent (EURx). Reverse transcription was performed using: 250 ng RNA, random primer and TranScriba reverse transcription kit (A&A Biotechnology) according to the manufacturer's instructions. The resulting cDNA was amplified with the qPCR RT HS-PCR Mix SYBR A kit (A&A Biotechnology) with complementary primers, respectively for:

TNC
(TN1: CCACGTACTTACCTGCACC (Seq ID No: 15),
TN2: CT-GATCTCTCCCTCATCTTC (Seq ID No: 16));
2′-5′-oligoadenylate synthetase 1
(OAS1F: CAGGAGCTCCAGGGCATAC (Seq ID No:17);
OAS1R: CATCCGCCTAGTCAAGCACT (Seq ID No: 18);
retinoic acid-inducible gene-I
(RIG1F: TTGGTATCTCCTAATCGCAAAAG (Seq ID No: 19);
RIG1IR: GGCAAGTCCCCCTGTAAAC (Seq ID No: 20));
beta actin
(ACB1: AGAGCTACGAGCTGCCTGAC (Seq ID No: 21);
ACB2: AG-CACTGTGTTGGCGTACAG (Seq ID No: 22));
hypoxanthine-guanine phosphoribosyltransferase 1
(HPRT1: TGACCTTGATT-TATTTTGCATAC (Seq ID No: 23),
HPRT2: CGAGCAAGACGTTCAGTCCT (Seq ID No: 24)).

The PCR reaction was carried out with initial denaturation at 95° C. for 3 min, followed by main denaturation at 95° C. for 15 s, hybridization at 62° C. for 30 s and amplicon extension at 72° C. for 20 s, followed by 39 cycles in the sequence: de-naturation-hybridization-extension, unless otherwise indicated.

### (VII) Evaluation of TNC Silencing

TNC silencing was determined as % of reduction in TNC expression relative to control with respect to the expression level of concurrently assayed reference genes ACTB and HPRT; where 100% indicates full silencing [more=better]. Statistical significance was determined by one-way ANOVA analysis, extended by Bonferroni's tst; * for p<0.033; ** for p<0.002; *** for p<0.001.

### (VIII) Evaluation of the Toxicity of LNP1 In Vivo

BALBc strain mice were used to evaluate the toxicity of the carrier comprising the proposed siRNAs silencing human TNC. This strain is the genetic background of the BALBc/nude mice that will be used in the target experiment of inoculating human glioblastoma multiforme cells and treating them with the proposed therapeutic molecule. BALBc/nude mice (CByJ.Cg-Foxn1nu/J) have no thymus gland and are unable to produce T lymphocytes and consequently have an impaired immune system and are ideally suited for xenograft (e.g. the introduction of human glioma cells into the mouse body).

BALBc mice were administered LNP1 molecules directly into the right cerebral ventricle during microsurgery in a stereotactic frame under general anaesthesia. LNP1 was administered at concentrations of 200 μg, 150 μg, 100 μg, 50 μg and 10 μg in a single volume of 10 μL as a bolus injection. This is to administer the entire dose of the therapeutic agent most efficiently to maximize biodistribution. The mice were then observed for 28 days for neurological disorders, apathy and body weight loss.

### Example 1. In-Silico Analysis of TNC Expression Level in Various Cancers

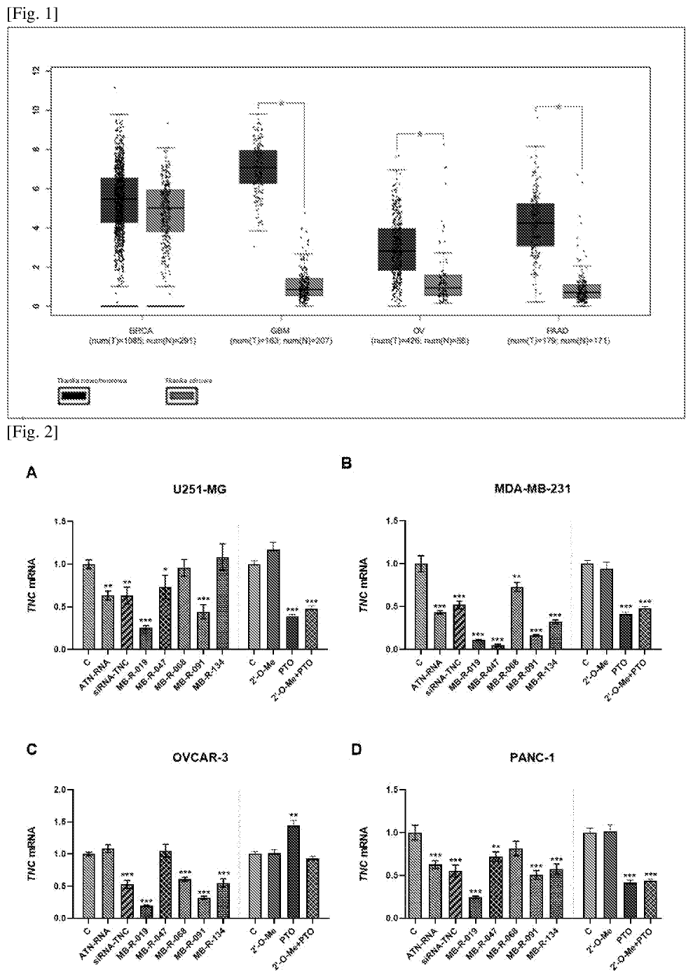

An analysis of TNC protein expression levels was performed on the GEPIA server [Tang et al. 2017] using cancer sequence data from The Cancer Genome Atlas (TCGA) database with a comparison to its expression corresponding to healthy tissues. Elevated TNC expression was observed in four cancer types, i.e. GBM, OV, BRCA, PAAD, compared to healthy patients ([FIG. 1]). Malignant gliomas including glioblastoma multiforme (GBM) and some breast (BRCA), ovarian (OV) and pancreatic (PAAD) cancers were demonstrated by in-silico analysis to exhibit elevated level of TNC, which can be used as a therapeutic target for the therapy of these cancers.

### Example 2. Generation of 21 nt siRNA-ATN within ATN-RNA and Analysis of their Effect on Reduction of TNC Level

siRNA-ATNs yielding lower toxicity and immunogenicity with greater efficacy in reducing TNC gene expression in glioma, ovarian, breast and pancreatic cancer cell lines were searched. For this purpose, 153 types of siRNA-ATN molecules with 21 nt in length were tested, which were formed by combining independently synthesized two single-stranded RNAs complementary to each other. Each siRNA-ATN was formed from the combination of a single-stranded RNA molecule with an effector (leader) sequence 5′->3′ and a single-stranded RNA molecule with a passenger sequence 5′->3′, formed on a matrix comprising a fragment corresponding to the ATN-RNA from the TNC gene. Each subsequent siRNA-ATN started one nucleotide further with respect to the ATN-RNA sequence (Table 2), the subsequent siRNA-ATNs were numbered as MB-R-001 to MB-R-153. To obtain the specific functionality of the synthesized siRNA molecules, they were chemically modified by introducing specific modifications indicated in Table 3. Their effect on the produced siRNAs was then confirmed, as shown in the following examples.

TABLE 2
ATN-RNA- sequence (consists of assembling an
effector sequence and a passenger sequence) -
refers to *binding site to TNC sequence 405-576 of
Seq ID No: 25.
Effector (leadER) sequence 5′→3′: (Seq ID No: 1)
caagcgacagaguggggugaacgccacccugccagaagagaaccagccag
ugguguuuaaccacguuuacaacaucaagcugccagugggaucccagugu
ucgguggaucuggagucagccaguggggagaaagaccuggcaccgccuuc
agagcccagcgaa
Passenger sequence 5′→3′: (Seq ID No: 2)
uucgcugggcucugaaggggugccaggucuuucuccccacuggcugacuc
cagauccaccgaacacugggaucccacuggcagcuugauguuguaaacgu
gguuaaacaccacuggcugguucucuucuggcaggguggcguucacccca
cucugucgcuug
*binding site to TNC was determined by the blastn algorithm based on the human transcript sequence

TABLE 3
Chemical modifications of siRNA molecules and their function.

| MODIFICATION | ACTION/FUNCTION OF THE MODIFICATION |
|---|---|
| 2′-O—Me | 3 bases of 2′O—Me were added at the 5′ and 3′ ends to |
| | increase resistance to nucleases and to increase the |
| | stability of the molecule itself |
| PTO-type | A sulfur atom was introduced in place of oxygen in the |
| bindings | phos-phodiester backbone to increase resistance to |
| | exonucleases |
| 2′-Fluoro RNA | Fluorine was substituted at the 2′ position of ribose |
| | to increase stability, resistance to chemical hydrolysis |
| | and the nucleases |
| 5′-E- | Added to mimic naturally occurring 5′-phosphate, |
| vinylphosphonate | which is rapidly metabolized by metabolic enzymes |
| 2′- | under in vivo conditions |
| methoxyuridine | |
| Cholesterol | The addition of lipophilic groups to the oligonucleotide |
| | increases cellular uptake/membrane permeation |
| [dTdT] | Deoxynucleotides [dTdT] were added at the 3′ end of |
| | the effector strand improving the specificity of binding |
| | of the effector strand to the target sequence and |
| | improving the stability of the molecule in the cell |

In view of the further planned tests, i.e. mass screening to determine the functionality of siRNAs from specific ATN regions on different cell lines, siRNAs for which deoxynucleotides [dTdT] were attached at the 3′ end of the effector strand during synthesis were used for further studies. The [dTdT] modification used minimises the so-called ‘off-target’ effect, i.e. non-specific silencing of transcripts, by promoting the formation of an RNA-induced silencing complex (RISC) with the effector strand. Furthermore, both the effector strand and the passenger siRNAs of the invention are complementary only to the TNC transcript. The glioma lines U118-MG and U251-MG, breast cancer MDA-MB-231, ovarian cancer OVCAR-3, and pancreatic cancer PANC-1 were used for transfection and determination of TNC gene transcript silencing level in vitro. Culture and transfection were performed as described above in (IV) in the presence of L2000 with ATN-RNA, the specific tested siRNA-ATN (in turn with constructs MB-R-001 to MB-R-153) and siRNA-TNC, which is siRNA of the non-ATN sequence. The siRNA-TNC was designed to be complementary to TNC mRNA sequence at positions 4625-4646, relative to the NM_002160.4 transcript (Table 2). After 48 h, RNA was isolated from the cells and RT-qPCR analysis was performed as described in (VI) above.

The efficacy of ATN-RNA and tested siRNA-ATN (sequentially from MB-R-001 to MB-R-153) and siRNA-TNC and examples of their 2′-O-Me modifications and PTO transfected into cancer cell lines were compared based on the TNC expression level study. Expression level in cells transfected with RNA at a concentration of 0.35 μg/mL (i.e. 0.175 μg RNA/well) were related to cells treated with carrier alone. Statistical significance was determined by one-way ANOVA analysis, extended by the Bonferroni's test; * for p<0.033; ** for p<0.002; *** for p<0.001. Of the tested siRNA-ATN molecules, five of them, i.e. MB-R-019, MB-R-047, MB-R-068, MB-R-091, MB-R-134, exhibited the highest silencing, but their effect varied depending on the cancer line used and the modification applied. The results obtained for selected siRNA-ATNs are shown in [FIG. 2] and in Table 5.

TABLE 4
Selected siRNA-ATNs directed against TNCs within the ATN area and
siRNA-TNCs of the non-ANT area and their sequences.

| | | | Effector (leader) sequence | | | |
|---|---|---|---|---|---|---|
| | Binding | | 5′→3′ | | | |
| | site | SITE | with an added modi- | | | |
| | to tnc^{$} | within | fication on 3′ - in this case | Seq ID | | |
| SeQUENCE | Seq | the | [dT][dT]. | No | PASSENGER | Seq |
| name | ID | ATN | The sequence given as | (without | sequence | ID |
| siRNA | No: 25 | sequence | Seq ID No has no modification | modification) | 5′→3′ | No |
| MB-R- | 423-443 | 19-39 | UCUUCUGGCAGGGU | 3 | GAACGCCACCCU | 14 |
| 019 | | | GGCGUUC[dT][dT] | | GCCAGAAGA | |
| MB-R- | 423-443 | 19-39 | UmCmUmUmCmUmGm | 3 | GAACGCCACCCU | 4 |
| 019- | | | GmCmAmGmGmGmUm | | GCCAGAAGA | |
| 2′O-Me | | | GmGmCmGmUmUmCm | | | |
| | | | [dT][dT] | | | |
| MB-R- | 423-443 | 19-39 | U*C*U*U*C*U*G*G*C | 3 | GAACGCCACCCU | 4 |
| 019- | | | *A*G*G*G*U*G*G*C* | | GCCAGAAGA | |
| PTO | | | G*U*U*C[dT][dT] | | | |
| MB-R- | 451-471 | 47-67 | AACGUGGUUAAACA | 5 | CCAGUGGUGUU | 6 |
| 047 | | | CCACUGG[dT][dT] | | UAACCACGUU | |
| MB-R- | 472-492 | 68-88 | CACUGGCAGCUUGA | 7 | UACAACAUCAA | 8 |
| 068 | | | UGUUGUA[dT][dT] | | GCUGCCAGUG | |
| MB-R- | 495-515 | 91-111 | AGAUCCACCGAACAC | 9 | AUCCCAGUGUUC | 10 |
| 091 | | | UGGGAU[dT][dT] | | GGUGGAUCU | |
| MB-R- | 538-558 | 134-154 | CUCUGAAGGCGGUG | 11 | GACCUGGCACCG | 12 |
| 134 | | | CCAGGUC[dT][dT] | | CCUUCAGAG | |
| siRNA- | 4625-4645 | — | UUAAGUUUCCAAUU | 13 | AACCUGAAAUU | 14 |
| TNC | | | UCAGGUU [dT][dT] | | GGAAACUUAA | |

^{$}relative to the human TNC sequence (Seq ID No: 25)
*PTO-type bindings
Am, Cm, Gm, Um - nucleotides modified with 2′-O-Me group

TABLE 5
TNC silencing as % of reduction in TNC expression relative to control [more = better].

| | Administered RNA molecule | | | | | | | | |
|---|---|---|---|---|---|---|---|---|---|
| | L2000- | L-2000- | L2000- | L2000- | L2000- | L2000- | L2000- | L2000- | L2000- |
| | ATN- | siRNA- | MB- | MB- | MB- | MB- | MB- | 019- | 019- |
| Cell line | RNA | TNC | R 019 | R 047 | R 068 | R 091 | R 134 | 2′OMe | PTO |
| U251-MG | 37% | 37% | 74% | 27% | 4% | 56% | * | * | 61% |
| (glioma) | | | | | | | | | |
| MDA-MB-231 | 57% | 48% | 89% | 95% | 27% | 84% | 68% | * | 59% |
| (breast | | | | | | | | | |
| cancer) | | | | | | | | | |
| OVCAR-3 | * | 47% | 80% | * | 44% | 68% | 45% | * | * |
| (ovarian | | | | | | | | | |
| cancer) | | | | | | | | | |
| PANC-1 | 37% | 45% | 75% | 28% | 19% | 49% | 43% | * | 58% |
| (pancreatic | | | | | | | | | |
| cancer) | | | | | | | | | |

* non-specific effect

The siRNAs listed in Tables 4 and 5, selected from the different siRNA-ATNs tested, have been shown to effectively reduce the INC gene transcript in specific cancer lines when introduced into cancer cells. The efficacy of the different siRNA molecules derived from ATN-RNA as well as those targeting the non-ATN-RNA sequence is not equivalent. MB-R-019, MB-R-091 and siRNA-TNC molecules show the highest efficacy and most potently reduce the INC gene transcript among the tested molecules in all studied cancer types. These siRNA molecules, depending on the cancer type, show higher (in the range of 38-99%) silencing than the ATN-RNA molecule (Table 5). In contrast, siRNA-ATN: MB-R-068 and MB-R-134 exhibited higher silencing capacity than the ATN-RNA molecule in breast cancer and ovarian cancer lines in the range of 68-96%.

The siRNAs used in this study (Table 4) comprised modifications in relation to the basic sequence obtained on the basis of ATN-RNA or INC of the non-ATN region involving the addition of [dTdT] nucleotides at the 3′ end of the effector strand. Such a modification improves the stability, efficiency and specificity of the siRNA molecule. As indicated above in Table 3, it is possible to produce siRNAs with other modifications to further increase the stability of the molecule and improve its efficiency in silencing the target sequence. However, as shown, the effect of reducing expression was dependent on the siRNA sequence and therefore the target sites with the highest TNC silencing potential located within the ATN sequence, not on the type of modification used for a particular siRNA molecule.

### Example 3. Cell Viability Analysis—Evaluation of RNA Cytotoxicity Upon Transfection with L2000

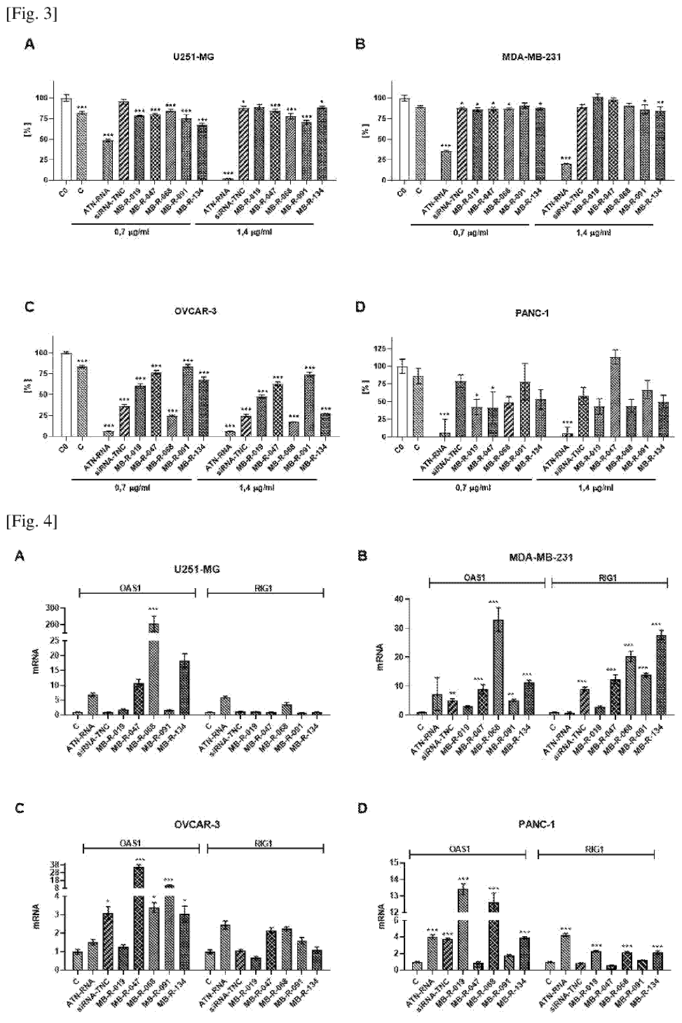

The procedure was started by passaging the cells from glioma (U251-MG), breast cancer (MDA-MB-231), ovarian (OVCAR-3) and pancreatic cancer (PANC-1) lines into 12-well culture plates. Cultures and transfection were performed as described above in (IV), for each RNA type at two RNA concentrations of 0.7 μg/mL and 1.4 μg/mL (0.35 μg RNA/well and 0.7 μg RNA/well, respectively), in at least three replicates. Cytotoxicity for a specific cell line was evaluated by MTT-based metabolic activity assay as described above in (V). Values obtained for cells treated with L2000, L2000 and ATN-RNA and a specific siRNA were related to untransfected cells, which were the control—C. The amount of L2000 used was constant at 1.75 μL/well. The statistical significance of the obtained results was determined by one-way ANOVA analysis, extended by Bonferroni's test; * for p<0.033; ** for p<0.002; *** for p<0.001. The obtained results are shown in [FIG. 3] and Table 6.

TABLE 6
Cytotoxicity evaluation - viability indicated as % of control; where 100% - corresponds
to the viability of untreated cells [more = better].

| | Transfected RNA complex | | | | | | |
|---|---|---|---|---|---|---|---|
| Cell line - | L2000- | L2000- | L2000- | L2000- | L2000- | L2000- | L2000- |
| administered | ATN- | siRNA- | MB- | MB- | MB- | MB- | MB- |
| concentration | RNA | TNC | R-019 | R-047 | R-068 | R-091 | R-134 |
| U-251 MG [0.7 μg/mL] | 49% | 95% | 78% | 80% | 85% | 76% | 68% |
| U-251 MG [1.4 μg/mL]. | 2% | 88% | 89% | 84% | 78% | 70% | 89% |
| MDA-MB-231 [0.7 μg/mL] | 36% | 87% | 86% | 87% | 87% | 91% | 87% |
| MDA-MB-231 [1.4 μg/mL] | 20% | 90% | 101% | 98% | 91% | 86% | 84% |
| OVCAR-3 [0.7 μg/mL] | 6% | 37% | 61% | 77% | 25% | 84% | 68% |
| OVCAR-3 [1.4 μg/mL] | 6% | 25% | 48% | 63% | 17% | 74% | 27% |
| PANC-1 [0.7 μg/mL] | 6% | 79% | 42% | 41% | 49% | 78% | 53% |
| PANC-1 [1.4 μg/mL] | 5% | 58% | 43% | 113% | 44% | 67% | 50% |

Unexpectedly, the L2000-ATN-RNA complex exhibited high cytotoxicity against all tested cell lines, increasing with the increase of the administered amount of ATN-RNA. At a concentration of 1.4 μg/mL for the U251-MG, OVCAR-3 and PANC-1 cell lines, complete necrosis of the tested cultures in vitro was demonstrated. The effect of the tested siRNAs on cell survival of the glioma or breast cancer lines did not exceed 50% in any case. In the ovarian cell line in the presence of the L2000 carrier, the molecules MB-R-019, MB-R-047 and MB-R-091 exhibited the lowest toxicity. In the pancreatic cell line in the presence of the L2000 carrier, the molecules siRNA-TNC, MB-R-091 and MB-R-134 exhibited the lowest toxicity. siRNA, regardless of type, exhibited much lower cytotoxicity compared to ATN-RNA introduced into cells at the same weight concentration.

### Example 4. Determination of Activation of Genes Associated with an Inflammatory Response—Evaluation of Immunogenicity at the Cellular Level with L2000 as a Carrier

To evaluate the immunogenicity of specific RNA constructs, i.e. ATN-RNA and selected siRNAs of glioma, breast cancer and ovarian cancer cells, their effects on the activation of inflammatory response genes, i.e. 2′-5′-oligoadenylate synthetase (OAS1) and retinoic acid-inducible gene I (RIG1), were studied. The procedure was started by passaging the cells from the cell line into 12-well culture plates. Cultures and transfection were performed as described above in (IV). Transfection of each cell line was performed with different RNAs: ATN-RNA at 0.35 μg/mL and siRNA at 0.35 μg/mL (0.175 μg RNA/well) using L2000 as a carrier. Cells were then harvested, RNA was isolated and RT-qPCR analysis was performed as described in (VI).

The effects of ATN-RNA, siRNA-ATN, and siRNA-TNC with a commercial carrier (L2000) on the induction of the immune response were compared by comparing the OAS1 and RIG1 expression levels in RNA-transfected cells and related to cells treated with carrier alone. Statistical significance was determined by one-way ANOVA analysis, extended by Bonferroni's test; * for p<0.033; ** for p<0.002; *** for p<0.001. The results are shown in [FIG. 4] and Table 7.

TABLE 7
Evaluation of the immunogenicity of individual RNAs expressed as % of
control against cells treated with carrier alone (taken as 100%) with
respect to expression of reference genes ACTB and HPRT [less = better].

| | Transfected RNA complex | | | | | | |
|---|---|---|---|---|---|---|---|
| | L2000- | L2000- | L2000- | L2000- | L2000- | L2000- | L2000- |
| Gene expression | ATN- | siRNA- | MB- | MB- | MB- | MB- | MB- |
| tested [%] - cell line | RNA | TNC | R-019 | R-047 | R-068 | R-091 | R-134 |
| OAS1 U-251 MG | 690% | 97% | 119% | 1100% | 20100% | 150% | 1830% |
| RIG1 U-251 MG | 600% | 120% | 108% | 92% | 340% | 74% | 101% |
| OAS1 MDA-MB-231 | 720% | 511% | 294% | 890% | 3300% | 510% | 1100% |
| RIG1 MDA-MB-231 | 76% | 895% | 283% | 1235% | 2000% | 1380% | 2770% |
| OAS1 OVCAR-3 | 150% | 310% | 125% | 3544% | 340% | 1100% | 300% |
| RIG1 OVCAR-3 | 240% | 105% | 68% | 210% | 220% | 160% | 110% |
| OAS1 PANC-1 | 405% | 86% | 230% | 59% | 217% | 118% | 215% |
| RIG1 PANC-1 | 425% | 377% | 1340% | 90% | 1260% | 180% | 392% |

The immunogenicity of individual siRNAs is not equivalent. It varies depending on the used cell line. The MB-R-019 molecule shows the lowest average immunogenicity in all tested cancer cell lines inducing more than 50% less increase in expression levels of the tested genes compared to ATN-RNA.

### Example 5. Comparison of TNC Silencing Efficiency for Administered Mixtures of Different siRNAs with L2000 as a Carrier

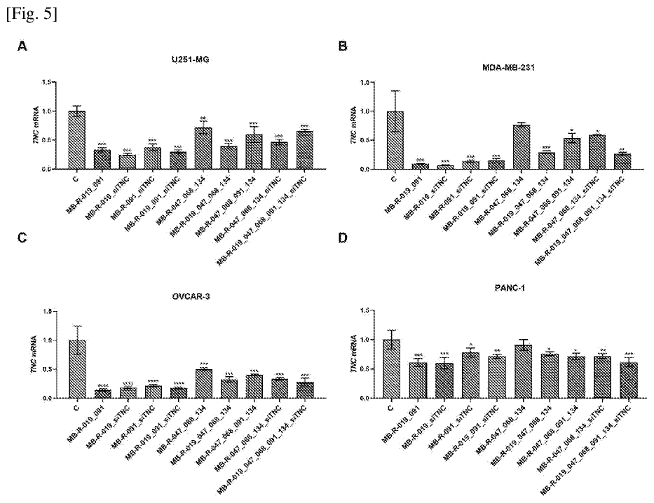

The effect on silencing TNC expression in glioma (U251-MG), breast cancer (MDA-MB-231), ovarian cancer (OVCAR-3) and pancreatic cancer (PANG-1) cells was studied by mixtures of different RNAs in L2000 at a concentration of 25 nM (0.35 ag/mL, 0.175 μg RNA/well). Some mixtures of siRNAs, especially two-component mixtures comprising MB-R-019, show better efficacy than single molecules in equal weight ratios and reduce TNC mRNA level in the range of 39-93% depending on the mixture composition and tested cell line ([FIG. 5], Table 8). At the same concentration, MB-R-019, MB-R-091 and siRNA-TNC molecules achieve efficiencies in the range of 37-89%. A synergistic effect of the used molecules is evident in the effect of the siRNA mixtures, which is impossible to predict.

TABLE 8
TNC silencing as % of reduction in TNC expression relative to control [more = better].

| | Transfected siRNA mixtures | | | | | | | | |
|---|---|---|---|---|---|---|---|---|---|
| | | | | | | 019 + | 047 + | 047 + | 019 + 047 + |
| | | | | 019 + | 047 + | 047 + | 068 + | 068 + | 068 + 91 + |
| | 019 + | 019 + | 091 + | 091 + | 068 + | 068 + | 091 + | 134 + | 134 + |
| Cell line | 091 | siTNC | siTNC | siTNC | 134 | 134 | 134 | siTNC | siTNC |
| U-251 MG | 83% | 83% | 74% | 78% | 52% | 77% | 61% | 64% | 68% |
| (glioma) | | | | | | | | | |
| MDA-MB-231 | 91% | 93% | 86% | 85% | 23% | 71% | 46% | 41% | 73% |
| (breast | | | | | | | | | |
| cancer) | | | | | | | | | |
| OVCAR-3 | 86% | 81% | 78% | 82% | 50% | 67% | 59% | 66% | 72% |
| (ovarian | | | | | | | | | |
| cancer) | | | | | | | | | |
| PANC-1 | 39% | 40% | 28% | 29% | 24% | 24% | 29% | 29% | 38% |
| (pancreatic | | | | | | | | | |
| cancer) | | | | | | | | | |

### Example 6. Cell Viability Analysis—Evaluation of ATN-RNA Cytotoxicity Upon Transfection with Different Lipid Carriers

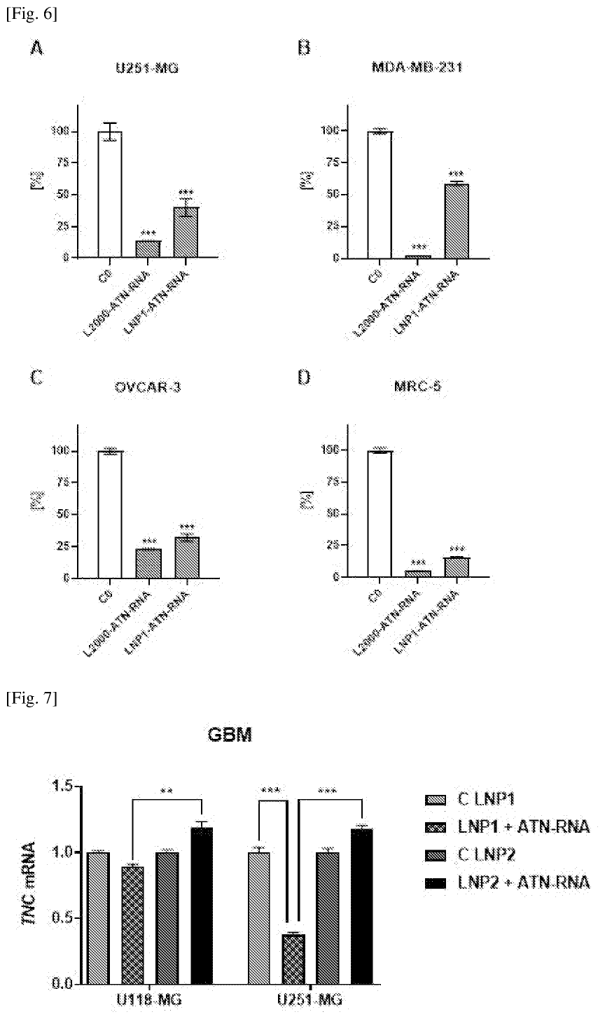

The procedure was started by passaging the cells from human glioma cell lines, breast cancer, ovarian lines and non-cancer cell lines into 12-well culture plates. Cultures and transfection were performed as described above in (IV). LNP1-ATN-RNA lipid nanoparticle complexes (LNP1 with ATN-RNA) were produced as described above in (II). ATN-RNA in a given lipid carrier was administered at a concentration of 12.5 nM (1.4 μg/mL, 0.7 μg RNA/well). Cytotoxicity to specific cell line was evaluated by an MIT-based metabolic activity assay as described above in (V). The toxicities of ATN-RNA in the presence of different lipid carriers were compared. Values obtained for ATN-RNA-treated cells were related to untreated control cells. Statistical significance was determined by one-way ANOVA analysis, extended by Bonferroni s test; **for p<0.001. The obtained results are shown in [FIG. 6] and Table 9.

TABLE 9
Evaluation of cytotoxicity - viability indicated as
% relative to control, where 100% - corresponds to
the viability of untreated cells [more = better].

| | Transfected complex: | Transfected complex: |
|---|---|---|
| Cell line | L2000-ATN-RNA | LNP1-ATN-RNA |
| U251-MG | 14% | 40% |
| MDA-MB-231 | 2% | 59% |
| OVCAR-3 | 23% | 32% |
| MRC-5 | 5% | 16% |

The results or a our cell lines indicate an average 3-fold higher survival of cells treated with the patented lipid complexes compared to L2000. This demonstrates the relatively lower toxicity of the patented transfection method.

### Example 7. Analysis of TNC Expression Level after Treatment with RNA Molecules in Different Carriers

Efficient carriers were searched to achieve stabilization of RNA molecules and their safe and efficient delivery to target cells for the best therapeutic results. For this purpose, lipid nanoparticle complexes with ATN-RNA and with various siRNAs were produced. An empty carrier was used as a control. According to the method presented in (II), cationic lipid nanoparticle LNP1 complexes and ionizable (neutral) lipid nanoparticle LNP2 complexes were produced.

### a) Comparison of TNC Silencing Level for ATN-RNA Administered in Cationic LNP1 and Neutral LNP2 Lipid Complexes

Cells from the U-118 MG and U-251 MG human glioma cell lines were passaged into 12-well culture plates. Cultures and transfection were performed as described above in (V). Transfection of each cell line was performed with LNP1-ATN-RNA and LNP2-ATN-RNA at a concentration of 1.4 μg/mL (0.7 μg RNA/well) for the U-118 MG line and 2.8 μg/mL (1.4 μg RNA/well) for the U-251 MG line. Cells were then harvested, RNA isolated and RT-qPCR analysis was performed to determine TNC expression level as described in (VI). The obtained results were related to cells treated with carrier alone. Statistical significance was determined by one-way ANOVA analysis, extended by Bonferroni's test; ** for p<0.01; *** for p<0.001. The obtained results are shown in [FIG. 7] and Table 10.

TABLE 10
Evaluation of TNC expression silencing relative to control
expressed in % [more = better].

| Glioma line | LNP1-ATN-RNA | LNP2-ATN-RNA |
|---|---|---|
| U118-MG | 11% | non-specific effect |
| U251-MG | 62% | non-specific effect |

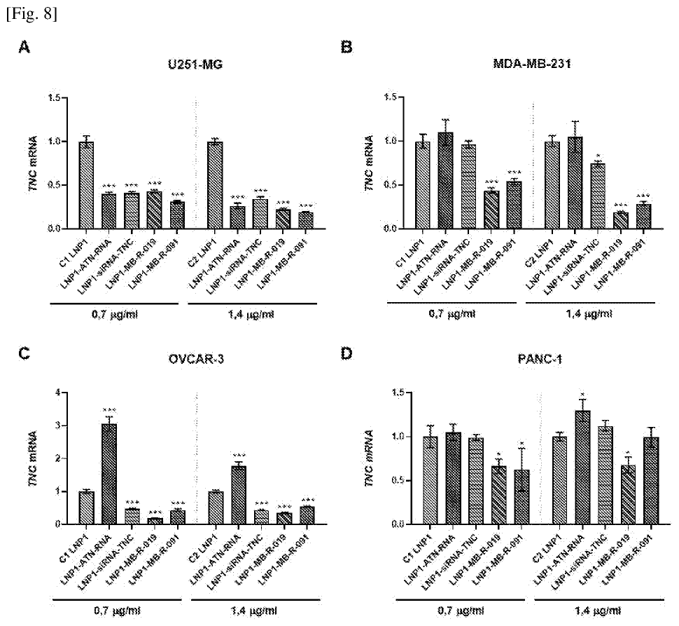

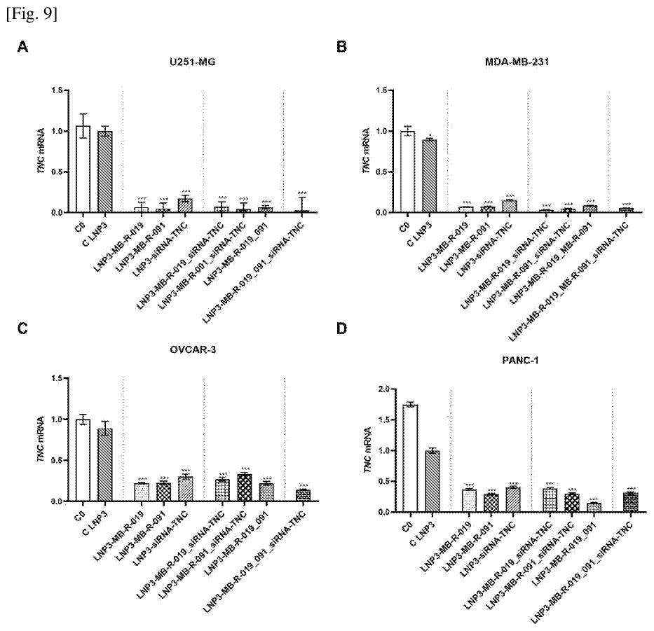

It was unexpectedly found that the use of the cationic carrier LNP1 led to more efficient silencing of the TNC gene compared to the neutral carrier LNP2, resulting in up to three times greater silencing of its expression. It was unexpectedly found that using the neutral (ionizable) carrier LNP2 only achieves non-specific results. Furthermore, it turned out that for TNC silencing, the type of carrier used is not irrelevant, and by using the wrong carrier, the opposite effect can be obtained. In the course of further work, the efficiency of TNC silencing using siRNA-ATN in the cationic carrier LNP1 was compared with the ionizable carrier LNP3. Compared to the LNP1 carrier, siRNA-ATN in the LNP3 carrier has a better efficiency for reducing TNC level ([FIG. 8], [FIG. 9], Table 11). In conclusion, it turned out that for TNC silencing, the type of carrier used is not irrelevant, and by using the wrong carrier, the opposite effect can be obtained. Thus, lipid complexes with a cationic (LNP1) or an ionizable (LNP3) carrier were used for further studies for different siRNAs.

TABLE 11
Evaluation of TNC expression silencing relative to control
expressed in % [more = better].

| Cell line | LNP1-MB-R-019 | LNP3-MB-R-019 |
|---|---|---|
| Glioma (U251-MG) | 57% | 82% |
| Breast cancer (MDA-MB-231) | 56% | 93% |
| Ovarian cancer (OVCAR-3) | 81% | 78% |
| Pancreatic cancer (PANC-1) | 33% | 63% |

B) Comparison of TNC Silencing for Administered Different siRNAs and ATN-RNAs in Cationic LNP1 Lipid Complexes

Cells from glioma (U251-MG), breast cancer (MDA-MB-231), ovarian cancer (OVCAR-3) and pancreatic cancer (PANC-1) cell lines were passaged into culture plates. Cultures and transfection were performed as described above in (IV) using LNP1 complexes as carriers of various RNAs. Transfection of each cell line was performed with LNP1-ATN-RNA, LNP1 with various selected siRNA-ATN and siRNA-TNC, administering each RNA at two concentrations of 50 nM (0.7 μg/mL, 0.35 μg RNA/well) and 100 nM (1.4 μg/mL, 0.7 μg RNA/well). Cells were then harvested, RNA isolated and RT-qPCR analysis was performed to determine TNC expression level as described in (VI). The results were related to cells treated with carrier alone and non-specific siRNA (MISSION® siRNA Universal Negative Control #2, cat. no. SIC002). Statistical significance was determined by one-way ANOVA analysis, extended by Bonferroni's test; * for p<0.033; ** for p<0.002; *** for p<0.001. The obtained results are shown in [FIG. 8] and Table 12.

TABLE 12
Evaluation of TNC expression silencing for the tested RNAs in
LNP1 lipid complexes relative to control, expressed in %, where
100% represents full silencing [more = better].

| | Transfected RNA complex in LNP1 and concentration used | | | | | | | |
|---|---|---|---|---|---|---|---|---|
| | 0.7 μg/mL | | | | 1.4 μg/mL | | | |
| | LNP1- | LNP1- | LNP1- | LNP1- | LNP1- | | LNP1- | LNP1- |
| | ATN- | siRNA- | MB- | MB- | ATN- | siRNA- | MB- | MB- |
| Cell line | RNA | TNC | R-019 | R-091 | RNA | TNC | R-019 | R-091 |
| U-251 MG | 60% | 59% | 57% | 69% | 74% | 66% | 78% | 81% |
| MDA-MB-231 | * | 4% | 56% | 47% | * | 26% | 81% | 71% |
| OVCAR-3 | * | 53% | 81% | 57% | * | 56% | 65% | 46% |
| PANC-1 | * | * | 33% | 38% | * | * | 32% | * |

* non-specific effect

The LNP N-MB-R-019 complex at a concentration of 5 nM shows the highest INC silencing efficiency in the tested cancer lines and reduces expression by as much as 33-81%, respectively.

C) Comparison of INC Silencing for Administered Mixtures of LNP3 with siRNA at Different Ratios

Proceeding as in B) above, the effect on the reduction ofINC mRNA level was studied in glioma (U251-MG), breast cancer (MDA-MB-231), ovarian cancer (OVCAR-3) and pancreatic cancer (PANC-1) cells by administering a mixture of LNP3 complexes with various RNAs at a concentration of 50 nM (0.7 ug/mL, 0.35 ug RNA/well). Some mixtures of siRNAs show better efficacy than single molecules in equal weight ratios. At a concentration of 0.7 μg/mL (0.35 μg RNA/well), a three-component mixture of LNP3-MB-R-019+MB-R-091+siRNA-TNC complexes shows the highest INC transcript reduction efficiency and exhibits efficiencies in the range of up to 97%. A synergistic effect of the molecules used is evident in the effect of the siRNA mixtures in LNP, which is impossible to predict. The obtained results are shown in [FIG. 9] and Table 13.

TABLE 13
Evaluation of TNC expression silencing in selected cell lines upon
transfection of mixtures of LNP3 complexes with different siRNAs compared
to siRNA-ATN and siRNA-TNC used alone, relative to control expressed
in %; where 100% represents full silencing [more = better].

| | | | | LNP3- | LNP3- | LNP3- | LNP3-MB- |
|---|---|---|---|---|---|---|---|
| | | | | MB- | MB- | MB- | R-019 + |
| | LNP3- | LNP3- | LNP3- | R - 019 + | R - 091 + | R-019 + | MB- |
| TNC silencing | MB- | MB- | siRNA- | siRNA- | siRNA- | MB- | R-091 + |
| [% of controls] | R-019 | R-091 | TNC | TNC | TNC | R-091 | siRNA-TNC |
| U251-MG | 82% | 82% | 65% | 79% | 77% | 81% | 81% |
| MDA-MB-231 | 93% | 93% | 85% | 97% | 95% | 91% | 94% |
| OVCAR-3 | 78% | 77% | 70% | 73% | 67% | 78% | 86% |
| PANC-1 | 63% | 71% | 59% | 61% | 70% | 85% | 69% |

* non-specific effect

### Example 8. Comparison of Toxicity of ATN-RNAs and Selected siRNAs AdMinistered at Different Concentrations in Cationic LNP1 Lipid Complexes

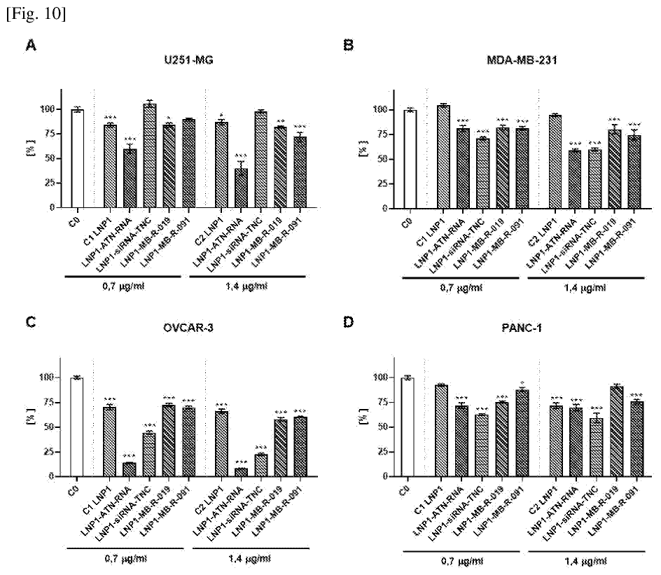

To evaluate the toxicity of the RNA complexes used in the LNP1 carrier, cell viability analyses were performed as in Example 3. The analysis was started by passaging the cells from glioma, breast cancer, ovarian cancer and pancreatic cancer lines into culture plates. Cultures and transfection were performed as described above in (IV), for each type of RNA at two concentrations of 0.7 μg/mL and 1.4 μg/mL, respectively (0.35 and 0.7 μg RNA/well). Cytotoxicity for a specific sample on a given cell line was evaluated by an MTT-based metabolic activity assay as described above in (V). Values obtained for LNP-treated cells with specific RNA were related to untreated cells. Statistical significance was determined by one-way ANOVA analysis, extended by Bonferroni s test; * for p<0.05; ** for p<0.01; *** for p<0.001. The obtained results are shown in [FIG. 10] and Table 14.

TABLE 14
Evaluation of cytotoxicity for tested RNAs in LNP1 lipid complexes - viability indicated as
% of control; where 100% - corresponds to the viability of untreated cells [more = better].

| | Transfected RNA complex in LNP1 and RNA concentration used | | | | | | | | | |
|---|---|---|---|---|---|---|---|---|---|---|
| | 0.7 μg/mL | | | | | 1.4 μg/mL | | | | |
| | | LNP1- | LNP1- | LNP1- | LNP1- | | LNP1- | LNP1- | LNP1- | LNP1- |
| | LNP- | ATN- | siRNA- | MB- | MB- | LNP- | ATN- | siRNA- | MB- | MB- |
| Cell line | C1 | RNA | TNC | R-019 | R-091 | C2 | RNA | TNC | R-019 | R-091 |
| U251-MG | 84% | 60% | 106% | 84% | 90% | 87% | 40% | 98% | 82% | 72% |
| MDA-MB-231 | 105% | 81% | 71% | 82% | 81% | 95% | 59% | 60% | 80% | 74% |
| OVCAR-3 | 71% | 14% | 44% | 73% | 70% | 66% | 8.5% | 23% | 58% | 61% |
| PANC-1 | 92% | 72% | 63% | 76% | 88% | 72% | 70% | 59% | 91% | 76% |

LNP1-siRNA complexes: LNP1-siRNA-TNC, LNP1-MB-R-019, LNP1-MB-R-091 show on average more than 30% less toxicity compared to LNP1-ATN-RNA complexes administered to the same cell lines. This means that the administration of selected TNC-lowering siRNAs in the form of cationic LNP1 lipid complexes with siRNAs will have a positive impact on patient survival, as the compositions comprising them are less toxic. Hence, compositions comprising LNP1-MB-R-019 and/or LNP1-MB-R-091 will be particularly preferable in cancer treatment.

### Example 9. Comparison of Toxicity of Selected siRNAs Administered at Different Concentrations in Ionizable LNP3 Lipid Complexes

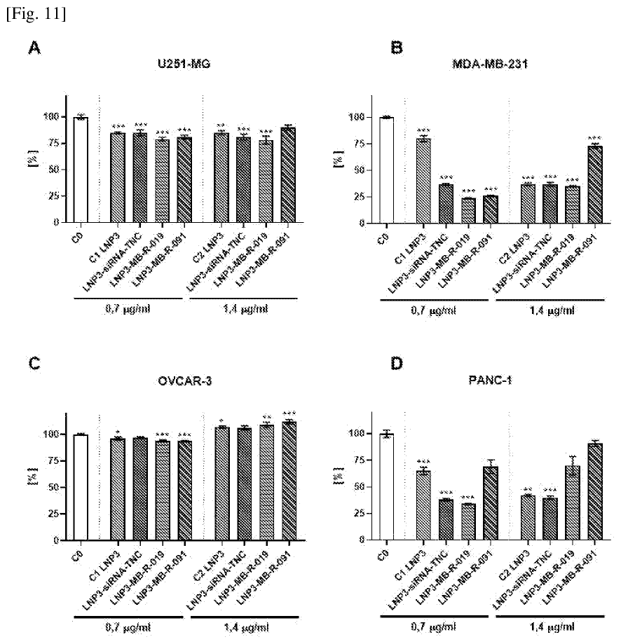

To evaluate the toxicity of the RNA complexes used in the LNP3 carrier, cell viability analyses were performed as in Example 3. The analysis was started by passaging the cells from glioma, breast cancer, ovarian cancer and pancreatic cancer lines into culture plates. Cultures and transfection were performed as described above in (IV), for each type of RNA at two concentrations of 0.7 μg/mL and 1.4 μg/mL, respectively (0.35 and 0.7 μg RNA/well). Cytotoxicity for a specific sample on a given cell line was evaluated by an MTT-based metabolic activity assay as described above in (V). Values obtained for LNP-treated cells with specific RNA were related to untreated cells. Statistical significance was determined by one-way ANOVA analysis, extended by Bonferroni's test; * for p<0.05; ** for p<0.01; *** for p<0.001. The obtained results are shown in [FIG. 11] and Table 15.

TABLE 15
Evaluation of cytotoxicity for tested RNAs in LNP3 lipid
complexes - viability indicated as % of control; where 100% -
corresponds to viability of untreated cells [more = better].

| | Transfected RNA complex in LNP3 and RNA concentration used | | | | | | | |
|---|---|---|---|---|---|---|---|---|
| | 0.7 μg/mL | | | | 1.4 μg/mL | | | |
| | | LNP3- | LNP3- | LNP3- | | LNP3- | LNP3- | LNP3- |
| | C1 | siRNA- | MB- | MB- | C2 | siRNA- | MB- | MB- |
| Cell line | LNP3 | TNC | R-019 | R-091 | LNP3 | TNC | R-019 | R-091 |
| U251-MG | 85% | 85% | 79% | 81% | 85% | 81% | 78% | 90% |
| MDA-MB-231 | 80% | 37% | 24% | 26% | 37% | 37% | 35% | 73% |
| OVCAR-3 | 96% | 97% | 94% | 94% | 107% | 106% | 109% | 112% |
| PANC-1 | 65% | 38% | 34% | 69% | 42% | 40% | 70% | 91% |

Administration of selected TNC-lowering siRNAs in the form of ionizable LNP3 lipid complexes with siRNAs does not show high toxicity relative to control, suggesting that their administration will be safe for patients. Compositions comprising LNP3-MB-R-019 and/or LNP3-MB-R-091 will be particularly preferable in cancer treatment.

### Example 10. Determination of Activation of Genes Associated with an Inflammatory Response—Evaluation of Immunogenicity of Cationic LNP1 Lipid Complexes—as RNA Carriers, at a Cellular Level

Evaluation of the immunogenicity of the different RNA constructs, i.e. ATN-RNA and selected siRNAs in the LNP1 carrier, on glioma, breast cancer, ovarian cancer and pancreatic cancer cell lines was determined by testing their effect on the activation of inflammatory response genes, i.e. 2′-5′-oligoadenylate synthetase (OAS1) and retinoic acid-inducible gene-I (RIG1), similarly to Example 4. Cultures and transfection were performed as described above in (IV). Transfection of each cell line was performed with different RNAs: using ATN-RNA at a concentration of 1.4 μg/mL and siRNA at a concentration of 1.4 μg/mL in LNP1 (0.75 μg RNA/well). Cells were then harvested, RNA isolated and RT-qPCR analysis of OAS1 and RIG1 expression was performed as described in (VI). The effects of ATN-RNA, tested siRNA-ATNs from the ATN area and siRNA-TNCs administered as cationic LNP1 lipid complexes on the induction of the immune response were compared by comparing the OAS1 and RIG1 expression levels in cells transfected with a specific LPN1 complex with RNA and related to cells treated with LNP1 carrier alone in an amount equivalent to the lipid nanoparticles contained in the complexes with RNA. Statistical significance was determined by one-way ANOVA analysis, extended by Bonferroni's test; * for p<0.033; ** for p<0.002; *** for p<0.001. The obtained results are shown in [FIG. 12] and Table 16.

TABLE 16
Evaluation of immunogenicity for the tested RNAs in LNP1 complexes,
against RIG1 and OAS1 gene expression; where 100% is the expression
in cells treated with LNP1 nanoparticles [less = better].

| | Evaluation of OAS1 expression | | | | Evaluation of RIGI expression | | | |
|---|---|---|---|---|---|---|---|---|
| | Transfected RNA complex | | | | Transfected RNA complex | | | |
| | LNP1- | LNP1- | LNP1- | LNP1- | LNP1- | LNP1- | LNP1- | LNP1- |
| | ATN- | siRNA- | MB- | MB- | ATN- | siRNA- | MB- | MB- |
| Cell line | RNA | TNC | R-019 | R-091 | RNA | TNC | R-019 | R-091 |
| U251-MG | 4800% | 950% | 900% | 120% | 5700% | 690% | 400% | 84% |
| MDA-MB-231 | 820% | 680% | 160% | 96% | 98% | 85% | 80% | 150% |
| OVCAR-3 | 2400% | 1800% | 240% | 140% | 340% | 590% | 140% | 140% |
| PANC-1 | 1450% | 4400% | 640% | 180% | 620% | 1000% | 140% | 190% |

It was unexpectedly found that LNP1-ATN-RNA and LNP1-siRNA-TNC exhibit a very strong increase in the inflammatory markers OAS1 and RIG1 in glioma, ovarian and pancreatic cancer lines and a high increase in RIG1 expression in breast cancer, and hence have lower therapeutic potential. It has also been shown that selected siRNA-ATNs (LNP1-MB-R-019 and LNP1-MB-R-091) administered in LPN1 induce up to 15 times lower immune responses compared to LNP1-ATN-RNA.

### Example 11. Evaluation of Stability of Naked RNA Molecules and in Complex with LNP1 in a Human Serum

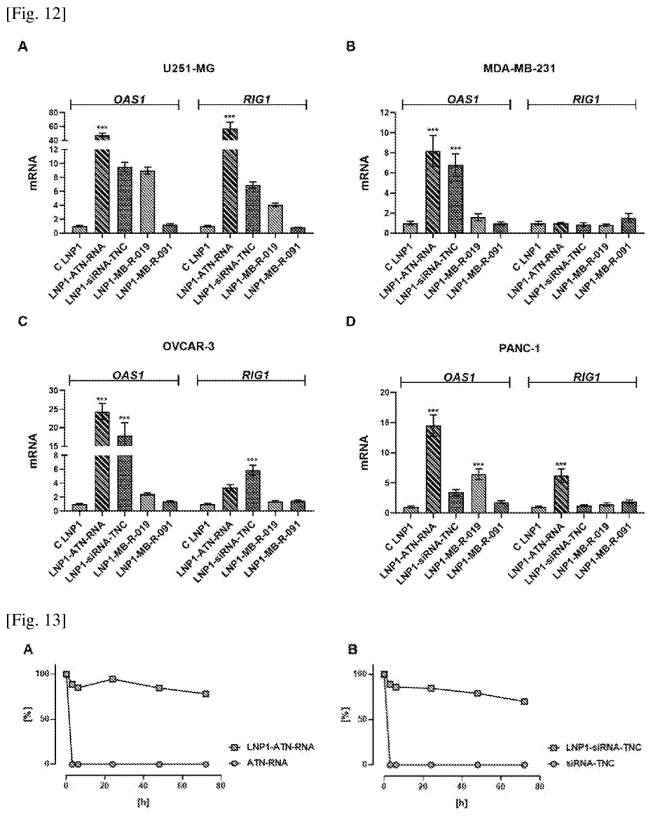

Determination of the stability of ATN-RNA and specific siRNA as well as the equimolar amount of the LNP1-ATN-RNA complex and specific LNP1-siRNA-TNC were performed as described in (III) above. The results of the densitometric signal intensity analysis for the individual samples are shown in [FIG. 13]. It turned out that the use of the LNP1 carrier protected the RNAs in the complex from degradation upon contact with cellular nucleases (>70% vs 0% within 72 h, LNP1-RNA vs unbound RNA, respectively).

### Example 12. Comparison of the Effects of Selected Modifications on siRNA Stability in a Human Serum

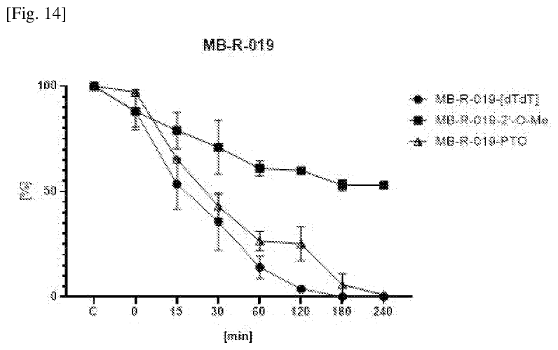

Determination of the stability of MB-R-019 was performed as described in (III) above. The results of the densitometric signal intensity analysis for each sample are shown in [FIG. 14]. It turned out that the type of chemical modification influenced the stability of the siRNA-ATN molecule in human serum. The attachment of a methyl group to the 2′OH ribose group protects the MB-R-019 molecule from degradation upon contact with cellular nucleases (>50% vs 0% within 240 min, respectively, MB-R-019-2′-O-Me vs MB-R-019-[dTdT]/MB-R-019-PTO).

### Example 13. Evaluation of the Toxicity of LNP1 and the LNP1-MB-R-019 Molecule Silencing Human TNC In Vivo

Evaluation of in vivo toxicity of the LNP1 carrier alone and of the LNP1-MB-R-019 complex was performed as described above in (VIII). The results, presented in Table 17, indicate that LNP1 was well tolerated by experimental animals when administered intraventricularly (into the brain) at a dose up to a maximum of 100 μg, which is 10 times the expected effective therapeutic dose (10 μg). Therapeutic siRNA-ATNs administered in the LNP1 carrier were also very well tolerated.

TABLE 17
Evaluation of the toxicity of LNP1 envelope-forming lipids and
LNP1-RNA complexes in vivo based on observed animal survival.

| | | LNP dose | Number | Number | Total |
|---|---|---|---|---|---|
| Administer | LNP dose | used | of live | of dead | number |
| edLNP | used | (mg/kg body | animals | animals | of |
| molecule | (μg/mouse) | weight) | [n/%] | [n/%] | animals |
| LNP1 | 200 | 10 | 1/50% | 1/50% | 2 |
| | 150 | 7.5 | 2/75% | 1/25% | 3 |
| | 100 | 5 | 3/100% | 0/0% | 3 |
| | 75 | 3.75 | 1/100% | 0/0% | 1 |
| LNP1-MB- | 50 (including | 2.5 (including | 3/100% | 0/0% | 3 |
| R-19 | 3 μg | 0.15 mg | | | |
| | siRNA) | siRNA) | | | |
| | 10 (including | 0.5 (including | 3/100% | 0/0% | 3 |
| | 0.6 μg | 0.03 mg | | | |
| | siRNA) | siRNA) | | | |

It turned out that therapeutic siRNA-ATN molecules according to the invention administered in vivo in the LNP1 carrier are very well tolerated and safe for mice.

### Example 14. Properties of LNP1 Complexes with siRNA Produced by the Method According to the Invention

LNP1-siRNA-ATN and LNP1-siRNA-TNC complexes were obtained by microfluidic mixing method as described above in (II). An N/P molar ratio of 8 was used based on literature data [Geall et al. 2012]. To confirm the homogeneity of the LNPs, their hydrodynamic diameter, polydispersity index and zeta potential were measured. The zeta potential is the potential between the dispersant and the fluid layer attached to the particle surface, used to evaluate dispersion stability. The incorporation efficiency of siRNA-TNC and the extent of its binding to LNP1 were also evaluated as in (II). The values obtained for the mentioned parameters are included in Table 18.

TABLE 18
Physicochemical properties of the resulting LNP1-
siRNA complexes according to the invention.

| | | | Zeta | Incorporation | Degree of |
|---|---|---|---|---|---|
| | zₐᵥ | | potential | efficiency | bonding |
| | [nm] | PDI* | [mV] | [%] | [%] |
| LNP1- | 92.3 ± | 0.093 ± | 3.5 ± | 81.5 ± | >99% |
| siRNA-TNC | 3.4 | 0.007 | 0.8 | 2.5% | |
| LNP1- | 121.1 ± | 0.045 ± | 1.43 ± | 86.1% ± | >99% |
| siRNA-019 | 6.05 | 0.013 | 1.04 | 2.5% | |

Z_{AV} (Z average) - hydrodynamic diameter
PDI - polydispersity index
*PDI <0.1 - superhomogeneous sample, PDI <0.25 - threshold value

The LNP-RNA complexes (LNP1 or LNP2 with a specific RNA: ATN-RNA, siRNA-ATN, siRNA-TNC) obtained by the microfluidic method yielded stable complexes with a size that guaranteed biological activity (below 100 nm), and a low polydispersity index (<0.1), indicating a very high degree of sample homogeneity. The application of the microfluidic method resulted in the obtained LNP-RNA complexes showing a number of advantages over the slow-mixing method [U.S. Ser. No. 11/026,894B2]: i.e. they were characterized by a smaller size of the obtained particles (92.3±3.4 nm vs 100±10 nm), high incorporation efficiency, a high degree of RNA binding, reproducibility and scalability of their production method. At the same time, the method used to produce LNP-RNA complexes according to the invention avoids the sonication and extrusion steps necessary in conventional LNP production methods, which negatively affect the stability of nucleic acids, causing their degradation and oxidation and often lead to formulation contamination. Furthermore, unlike the slow-mixing method known from the state of the art, the microfluidic method used not only yields high quality LNP-RNA complexes, but also allows the synthesis process to be easily transferred from the laboratory scale to the industrial scale.

### Sequence Listing Free Text

Seq ID Nos: 15-24 sequences presented in electronic version (and in Examples, Materials and methods (IV)) Seq ID No: 25 presented electronically and available under NM_002160.4)

| <110> Medicofarma Biotech | |
|---|---|
| <120> siRNA molecule against human tenascin-C (TNC) | |
| <130> PPL/1328/AGR | |
| <160> 24 | |
| <170> PatentIn version 3.5 | |
| <210> 1 | |
| <211> 163 | |
| <212> RNA | |
| <213> Homo sapiens | |
| <400> 1 | |
| caagcgacag agugggguga acgccacccu gccagaagag aaccagccag ugguguuuaa | 60 |
| ccacguuuac aacaucaagc ugccaguggg aucccagugu ucgguggauc uggagucagc | 120 |
| caguggggag aaagaccugg caccgccuuc agagcccagc gaa | 163 |
| <210> 2 | |
| <211> 163 | |
| <212> RNA | |
| <213> homo sapiens | |
| <400> 2 | |
| uucgcugggc ucugaaggcg gugccagguc uuucucccca cuggcugacu ccagauccac | 60 |
| cgaacacugg gaucccacug gcagcuugau guuguaaacg ugguuaaaca ccacuggcug | 120 |
| guucucuucu ggcagggugg cguucacccc acucugucgc uug | 163 |
| <210> 3 | |
| <211> 21 | |
| <212> RNA | |
| <213> artificial | |
| <220> | |
| <223> synthetic ssRNA | |
| <400> 3 | |
| ucuucuggca ggguggcguu c | 21 |
| <210> 4 | |
| <211> 21 | |
| <212> RNA | |
| <213> artificial | |
| <220> | |
| <223> synthetic ssRNA | |
| <400> 4 | |
| gaacgccacc cugccagaag a | 21 |
| <210> 5 | |
| <211> 21 | |
| <212> RNA | |
| <213> artificial | |
| <220> | |
| <223> synthetic ssRNA | |
| <400> 5 | |
| aacgugguua aacaccacug g | 21 |
| <210> 6 | |
| <211> 21 | |
| <212> RNA | |
| <213> artificial | |
| <220> | |
| <223> synthetic ssRNA | |
| <400> 6 | |
| ccaguggugu uuaaccacgu u | 21 |
| <210> 7 | |
| <211> 21 | |
| <212> RNA | |
| <213> artificial | |
| <220> | |
| <223> synthetic ssRNA | |
| <400> 7 | |
| cacuggcage uugauguugu a | 21 |
| <210> 8 | |
| <211> 21 | |
| <212> RNA | |
| <213> artificial | |
| <220> | |
| <223> synthetic ssRNA | |
| <400> 8 | |
| uacaacauca agcugccagu g | 21 |
| <210> 9 | |
| <211> 21 | |
| <212> RNA | |
| <213> artificial | |
| <220> | |
| <223> synthetic ssRNA | |
| <400> 9 | |
| agauccaccg aacacuggga u | 21 |
| <210> 10 | |
| <211> 21 | |
| <212> RNA | |
| <213> artificial | |
| <220> | |
| <223> synthetic ssRNA | |
| <400> 10 | |
| aucccagugu ucgguggauc u | 21 |
| <210> 11 | |
| <211> 21 | |
| <212> RNA | |
| <213> synthetic ssRNA | |
| <400> 11 | |
| cucugaaggc ggugccaggu c | 21 |
| <210> 12 | |
| <211> 21 | |
| <212> RNA | |
| <213> artificial | |
| <220> | |
| <223> synthetic ssRNA | |
| <400> 12 | |
| gaccuggcac cgccuucaga g | 21 |
| <210> 13 | |
| <211> 21 | |
| <212> RNA | |
| <213> artificial | |
| <220> | |
| <223> synthetic ssRNA | |
| <400> 13 | |
| uuaaguuucc aauuucaggu u | 21 |
| <210> 14 | |
| <211> 21 | |
| <212> RNA | |
| <213> artificial | |
| <220> | |
| <223> synthetic ssRNA | |
| <400> 14 | |
| aaccugaaau uggaaacuua a | 21 |

<210> 15
<211> 19
<212> DNA
<213> artificial
<220>

### CITATION LIST

Akinc A et al. The Onpattro story and the clinical translation of nanomedicines containing nucleic acid-based drugs. Nat. Nanotechnol. 2019; 14: 1084-1087.
Balasenthil S et al. A migration signature and plasma biomarker panel for pancreatic adenocarcinoma. Cancer Prev Res (Phila). 2011; 4:137-49.
Balasenthil S et al. A plasma biomarker panel to identify surgically resectable early-stage pancreatic cancer. J Natl Cancer Inst. 2017; 109(8): 1-10.
Behrem S et al. Distribution pattern of tenascin-C in glioblastoma: correlation with angiogenesis and tumor cell proliferation. Pathol Oncol Res. 2005; 11: 229-35.
Cai J et al. Tenascin-C modulates cell cycle progression to enhance tumour cell proliferation through akt/foxo1 signalling in pancreatic cancer. JCancer 2018; 9: 4449-4462.
Chiquet-Ehrismann R and Chiquet M. Tenascins: regulation and putative functions during pathological stress. J Pathol 2003; 200: 488-99.
Chonn A et al. The role of surface charge in the activation of the classical and al-ternative pathways of complement by liposomes. J Immunol. 1991; 146: 4234-41.
Dammes N et al. Paving the road for RNA therapeutics. Trends Pharmacol Sci. 2020; 41: 755-775.
Esposito I et al. Tenascin C and annexin II expression in the process of pancreatic carcinogenesis. J Pathol. 2006; 208: 673-85.
Fu Z et al. In vivo self-assembled small RNAs as a new generation of RNAi therapeutics. Cell Res. 2021; 31: 631-648.
Furusawaa Y et al. Effects of therapeutic ultrasound on the nucleus and genomic DNA. Ultrasonics Sonochemistry 2014; 21: 2061-2068
Geall A J et al. Nonviral delivery of self-amplifying RNA vaccines. Proceedings of the National Academy of Sciences. 2012; 109: 14604-14609.
Grabowska, M. et al. Nano-mediated delivery of double-stranded RNA for gene therapy of glioblastoma multiforme. PLoS ONE. 2019; 14(3): 1-21.
Grzelinski Mariusz et al. RNA interference-mediated gene silencing of pleiotrophin through polyethylenimine-complexed small interfering RNAs in vivo exerts antitumoral effects in glioblastoma xenografts. Human Gene Therapy. 2006; 17: 751-766.
Guttery D S et al. Association of invasion-promoting tenascin-C additional domains with breast cancers in young women. Breast Cancer Res. 2010; 12: R57.
Halder J et al. Focal adhesion kinase targeting using in vivo short interfering RNA delivery in neutral liposomes for ovarian carcinoma therapy. Clin Cancer Res. 2006; 12: 4916-24.
Hu B et al. Therapeutic siRNA: state of the art. Signal Transduction and Targeted Therapy. 2020; 5:101.
Jahkola T et al. Tenascin-C expression in invasion border of early breast cancer: a predictor of local and distant recurrence. Br J Cancer. 1998; 78:1507-13.
Jahn A et al. Controlled Vesicle Self-Assembly in Microfluidic Channels with Hydrodynamic Focusing. J. Am. Chem. Soc. 2004; 126: 2674-2675.
Jallo G I et al. Tenascin-C expression in the cyst wall and fluid of human brain tumors correlates with angiogenesis. Neurosurgery 1997; 5: 1052-9.
Leins A et al. Expression of tenascin-C in various human brain tumors and its relevance for survival in patients with astrocytoma. Cancer 2003; 98: 2430-9.
Li Z L et al. Autophagy deficiency promotes triple-negative breast cancer resistance to T cell-mediated cytotoxicity by blocking tenascin-C degradation. Nat Commun 2020; 11,3806.
Lieleg O et al. Selective filtering of particles by the extracellular matrix: an electrostatic bandpass. Biophysical Journal. 2009; 97: 1569-1577.
Liot S et al. Stroma involvement in pancreatic ductal adenocarcinoma: an overview focusing on extracellular matrix proteins. Front. Immunol. 2021; 12: 612271.
Ma K K et al. Triple Negative Status is a Poor Prognostic Indicator in Chinese Women with Breast Cancer: a Ten Year Review. Asian Pacific Journal of Cancer Prevention 2012; 13: 2109-2114.
MacLachlan I. Liposomal formulations for nucleic acid delivery. Antisense Drug Technol. Princ. Strat. Appl. 2007; 2: 237-270.
Martina et al. Tenascin-W is a specific marker of glioma-associated blood vessels and stimulates angiogenesis in vitro. FASEB J. 2010; 24: 778-787.
Ming X et al. Prognostic Role of Tenascin-C for Cancer Outcome: A Meta-Analysis. Technology in Cancer Research & Treatment. 2019; 18: 1-9.
Nie S et al. Tenascin-C: A Novel Candidate Marker for Cancer Stem Cells in Glioblastoma Identified by Tissue Microarrays. J. Proteome Res. 2015; 14: 814-822.
Ortega-Berlanga B et al. Recent Advances in the Use of Lipid-Based Nanoparticles Against Glioblastoma Multiforme. Arch. Immunol. Ther. Exp. 2021; 69:8.
Oskarsson T et al. Breast cancer cells produce tenascin C as a metastatic niche component to colonize the lungs. Nat Med 2011; 17: 867-874.
Pas J et al. Analysis of structure and function of tenascin-C. Int J Biochem Cell Biol. 2006; 38: 1594-602.
Qian S et al. Exosomal Tenascin-c induces proliferation and invasion of pancreatic cancer cells by WNT signaling. OncoTargets and Therapy 2019; 12: 3197-3205.
Reardon D A et al. A pilot study: 131I-Antitenascin monoclonal antibody 81c6 to deliver a 44-Gy resection cavity boost. Neuro Oncology 2008; 10: 182-189.
Reardon D A et al. Phase II trial of murine (131) I-labeled antitenascin monoclonal antibody 81C6 administered into surgically created resection cavities of patients with newly diagnosed malignant gliomas. J Clin Oncol. 2002; 20: 1389-1397.
Roces C. B. et al. Manufacturing Considerations for the Development of Lipid Nanoparticles Using Microfluidics. Pharmaceutics 2020; 11: 1095.
Rolle K et al. Promising human brain tumors therapy with interference RNA in-tervention (iRNAi). Cancer Biology&Therapy 2010; 9:5: 396-406.
Semple S C et al. Efficient encapsulation of antisense oligonucleotides in lipid vesicles using ionizable aminolipids: Formation of novel small multilamellar vesicle structures. Biochim. Biophys. Acta (BBA) Biomembr. 2001; 1510: 152-166.
Schlich M et al. Cytosolic delivery of nucleic acids: The case of ionizable lipid nanoparticles. Bioeng Transl Med. 2021; 20: e10213.
Tang Z. et al. GEPIA: a web server for cancer and normal gene expression profiling and interactive analyses. Nucleic Acids Res. 2017; 45: 98-102.
Tas F et al. Clinical significance of serum protease-activated receptor-1 levels in gastric cancer patients. Biomed Rep. 2016; 4: 489-492.
Wawrzyniak D et al. Down-regulation of tenascin-C inhibits breast cancer cells development by cell growth, migration, and adhesion impairment. PLoS ONE 2020; 15(8): 1-25.
Wilson K E et al. Expression of the extracellular matrix protein tenascin in malignant and benign ovarian tumours. Br J Cancer 1996; 74: 999-1004.
Wilson K E et al. Regulation and function of the extracellular matrix protein tenascin-C in ovarian cancer cell lines. Br J Cancer. 1999; 80: 685-692.
Yang et al. Tenascin C is a prognostic determinant and potential cancer-associated fibroblasts marker for breast ductal carcinoma. Experimental and Molecular Pathology 2017; 102: 262-267.
Yoshida J et al. Human gene therapy for malignant gliomas (glioblastoma multiforme and anaplastic astrocytoma) by in vivo transduction with human interferon b gene using cationic liposomes. Human Gene Therapy 2004; 15: 77-86.
Zukiel R et al. Suppression of human brain tumor with interference RNA specific for tenascin-C. Cancer Biol Ther. 2006; 5: 1002-7.

1-16. (canceled)

17. A siRNA molecule against a human tenascin-C (TNC) transcript sequence (disclosed in the NCBI database as NM_002160.4, Seq ID No: 25) for silencing of human TNC expression, characterized in that
(i) it is at least 80% identical with a complementary sequence of the TNC mRNA in a 423-443 nucleotide region of Seq ID No: 25, and/or comprises a molecule with a sequence at least 80% identical with Seq ID No: 3;
(iv) it is at least 80% identical with a complementary sequence of the TNC mRNA in a 495-515 nucleotide region of Seq ID No: 25, and/or comprises a molecule with a sequence at least 80% identical with Seq ID No: 9;
(vi) it is at least 80% identical with a complementary sequence of the TNC mRNA in a 4625-4645 nucleotide region of Seq ID No: 25, and/or comprises a molecule with a sequence at least 80% identical with Seq ID No: 13.

18. The siRNA molecule according to claim 17, characterized in that
(i) it is at least 85%, more preferably 90%, more preferably 95% identical with the complementary sequence of the TNC mRNA in the 423-443 nucleotide region of Seq ID No: 25, and/or comprises a molecule at least 85%, more preferably 90%, more preferably 95% identical with Seq ID No: 3;
(iv) it is at least 85%, more preferably 90%, more preferably 95% identical with the complementary sequence of the TNC mRNA in the 495-515 nucleotide region of Seq ID No: 25, and/or comprises a molecule at least 85%, more preferably 90%, more preferably 95% identical with Seq ID No: 9;
(vi) it is at least 85%, more preferably 90%, more preferably 95% identical with the complementary sequence of the TNC mRNA in the 4625-4645 nucleotide region of Seq ID No: 25, and/or comprises a molecule at least 85%, more preferably 90%, more preferably 95% identical with Seq ID No: 13.

19. The siRNA molecule according to claim 17, characterized in that the siRNA molecule comprises a sequence of at least 21 nucleotides in length.

20. The siRNA molecule according to claim 17, characterized in that the siRNA molecule comprises a sequence between 21-30 nucleotides in length.

21. The siRNA molecule according to claim 17, characterized in that the siRNA molecule comprises a sequence of at least 21 nucleotides in length, wherein
(i) it is at least 85%, more preferably 96%, more preferably 99%, more preferably 100% identical with the complementary sequence of the TNC mRNA in the 423-443 nucleotide region of Seq ID No: 25, and/or comprises a molecule at least 85%, more preferably 90%, more preferably 96%, more preferably 99%, more preferably 100% identical with Seq ID No: 3, most preferably is a molecule with the sequence of Seq ID No: 3;
(iv) it is at least 85%, more preferably 96%, more preferably 99%, more preferably 100% identical with the complementary sequence of the TNC mRNA in the 495-515 nucleotide region of Seq ID No: 25, and/or comprises a molecule at least 85%, more preferably 90%, more preferably 96%, more preferably 99%, more preferably 100% identical with Seq ID No: 9; most preferably is a molecule with the sequence of Seq ID No: 9;
(vi) it is at least 85%, more preferably 96%, more preferably 99%, more preferably 100% identical with the complementary sequence of the TNC mRNA in the 4625-4645 nucleotide region of Seq ID No: 25, and/or comprises a molecule at least 85%, more preferably 90%, more preferably 96%, more preferably 99%, more preferably 100% identical with Seq ID No 13, most preferably is a molecule with the sequence of Seq ID No 13.

22. The siRNA molecule according to claim 17, characterized in that the siRNA molecule is in the form of a double-stranded RNA (dsRNA) molecule with or without from 2 to 4 nucleotide overhangs, wherein the dsRNA consists of a single-stranded ssRNA of an effector molecule and an ssRNA passenger molecule and wherein the duplex region being between 19-30 nucleotides.

23. The siRNA molecule according to claim 17, characterized in that the siRNA molecule is an siRNA molecule selected from
(i) MB-R-019 being a duplex of the effector sequence of Seq ID No: 3 with the passenger sequence of Seq ID No: 4;
(iv) MB-R-091 being a duplex of the effector sequence of Seq ID No: 9 with the passenger sequence of Seq ID No: 10;
(vi) siRNA-TNC being a duplex of the effector sequence of Seq ID No: 13 with the passenger sequence of Seq ID No: 14.

24. The siRNA molecule according to claim 17, characterized in that the siRNA molecule comprises at least one chemically modified nucleotide and/or at least one modification selected from 2′-O-Me modification, PTO-type binding, 2′-Fluoro RNA modification, 5′-E vinylphosphonate 2′-methoxyuridine modification, modification with cholesterol, modification by deoxynucleotide attachment [dTdT].

25. A pharmaceutical composition comprising at least one siRNA as defined in claim 17, and a pharmaceutically acceptable carrier, vehicle or excipient for use in a treatment and/or prevention of the development of cancer characterized by increased TNC expression in a human, wherein the treatment and/or prevention occurs by inhibiting TNC expression.

26. The pharmaceutical composition according to claim 25, characterized in that the cancer with increased TNC expression is selected from glioma, breast cancer, ovarian cancer, and pancreatic cancer.

27. The pharmaceutical composition according to claim 25,
wherein the pharmaceutical composition comprises at least one siRNA molecule selected from the group comprising:
MB-R-019 being a duplex of the effector sequence of Seq ID No 3 with the passenger sequence of Seq ID No 4,
MB-R-091 being a duplex of the effector sequence of Seq ID No 9 with the passenger sequence of Seq ID No 10,
siRNA-TNC being a duplex of the effector sequence of Seq ID No 13 with the passenger sequence of Seq ID No 14,
or any mixture thereof.

28. The pharmaceutical composition according to claim 25, characterized in that the siRNA comprises at least one modification selected from 2′-O-Me modification, PTO-type binding, 2′-Fluoro RNA modification, 5′-E vinylphosphonate 2′-methoxyuridine modification, modification with cholesterol, modification by deoxynucleotide attachment [dTdT] at the 3′ end of the effector strand.

29. The pharmaceutical composition according to claim 25, characterized in that the pharmaceutical composition comprises a mixture of at least two randomly selected siRNA molecules in any molar ratio of 1 to 10 relative to the siRNA molecules.


SEQUENCE LISTING (25 sequences)

SEQ ID NO: 1 - RNA, 163 nt, Homo sapiens
  source 1..163: mol_type = genomic RNA
caagcgacagagtggggtgaacgccaccctgccagaagagaaccagccagtggtgtttaaccacgtttacaacatcaagctgccagtgggatcccagtgttcggtggatctggagtcagccagtggggagaaagacctggcaccgccttcagagcccagcgaa

SEQ ID NO: 2 - RNA, 163 nt, Homo sapiens
  source 1..163: mol_type = genomic RNA
ttcgctgggctctgaaggcggtgccaggtctttctccccactggctgactccagatccaccgaacactgggatcccactggcagcttgatgttgtaaacgtggttaaacaccactggctggttctcttctggcagggtggcgttcaccccactctgtcgcttg

SEQ ID NO: 3 - RNA, 21 nt, synthetic construct
  misc_feature 1..21: note = synthetic ssRNA
  source 1..21: mol_type = other RNA
tcttctggcagggtggcgttc

SEQ ID NO: 4 - RNA, 21 nt, synthetic construct
  misc_feature 1..21: note = synthetic ssRNA
  source 1..21: mol_type = other RNA
gaacgccaccctgccagaaga

SEQ ID NO: 5 - RNA, 21 nt, synthetic construct
  misc_feature 1..21: note = synthetic ssRNA
  source 1..21: mol_type = other RNA
aacgtggttaaacaccactgg

SEQ ID NO: 6 - RNA, 21 nt, synthetic construct
  misc_feature 1..21: note = synthetic ssRNA
  source 1..21: mol_type = other RNA
ccagtggtgtttaaccacgtt

SEQ ID NO: 7 - RNA, 21 nt, synthetic construct
  misc_feature 1..21: note = synthetic ssRNA
  source 1..21: mol_type = other RNA
cactggcagcttgatgttgta

SEQ ID NO: 8 - RNA, 21 nt, synthetic construct
  misc_feature 1..21: note = synthetic ssRNA
  source 1..21: mol_type = other RNA
tacaacatcaagctgccagtg

SEQ ID NO: 9 - RNA, 21 nt, synthetic construct
  misc_feature 1..21: note = synthetic ssRNA
  source 1..21: mol_type = other RNA
agatccaccgaacactgggat

SEQ ID NO: 10 - RNA, 21 nt, synthetic construct
  misc_feature 1..21: note = synthetic ssRNA
  source 1..21: mol_type = other RNA
atcccagtgttcggtggatct

SEQ ID NO: 11 - RNA, 21 nt, synthetic construct
  source 1..21: mol_type = other RNA
ctctgaaggcggtgccaggtc

SEQ ID NO: 12 - RNA, 21 nt, synthetic construct
  misc_feature 1..21: note = synthetic ssRNA
  source 1..21: mol_type = other RNA
gacctggcaccgccttcagag

SEQ ID NO: 13 - RNA, 21 nt, synthetic construct
  misc_feature 1..21: note = synthetic ssRNA
  source 1..21: mol_type = other RNA
ttaagtttccaatttcaggtt

SEQ ID NO: 14 - RNA, 21 nt, synthetic construct
  misc_feature 1..21: note = synthetic ssRNA
  source 1..21: mol_type = other RNA
aacctgaaattggaaacttaa

SEQ ID NO: 15 - DNA, 19 bp, synthetic construct
  misc_feature 1..19: note = ssDNA TN1
  source 1..19: mol_type = other DNA
ccacgtacttacctgcacc

SEQ ID NO: 16 - DNA, 20 bp, synthetic construct
  misc_feature 1..20: note = ssDNA TN2
  source 1..20: mol_type = other DNA
ctgatctctccctcatcttc

SEQ ID NO: 17 - DNA, 19 bp, synthetic construct
  misc_feature 1..19: note = ssDNA OAS1F
  source 1..19: mol_type = other DNA
caggagctccagggcatac

SEQ ID NO: 18 - DNA, 20 bp, synthetic construct
  misc_feature 1..20: note = ssDNA OAS1R
  source 1..20: mol_type = other DNA
catccgcctagtcaagcact

SEQ ID NO: 19 - DNA, 20 bp, synthetic construct
  misc_feature 1..20: note = ssDNA OAS1R
  source 1..20: mol_type = other DNA
catccgcctagtcaagcact

SEQ ID NO: 20 - DNA, 19 bp, synthetic construct
  misc_feature 1..19: note = ssDNA RIG1R
  source 1..19: mol_type = other DNA
ggcaagtccccctgtaaac

SEQ ID NO: 21 - DNA, 20 bp, synthetic construct
  misc_feature 1..20: note = ssDNA ACB1
  source 1..20: mol_type = other DNA
agagctacgagctgcctgac

SEQ ID NO: 22 - DNA, 20 bp, synthetic construct
  misc_feature 1..20: note = ssDNA ACB2
  source 1..20: mol_type = other DNA
agcactgtgttggcgtacag

SEQ ID NO: 23 - DNA, 23 bp, synthetic construct
  misc_feature 1..23: note = ssDNA HPRT1
  source 1..23: mol_type = other DNA
tgaccttgatttattttgcatac

SEQ ID NO: 24 - DNA, 20 bp, synthetic construct
  misc_feature 1..20: note = ssDNA HPRT2
  source 1..20: mol_type = other DNA
cgagcaagacgttcagtcct

SEQ ID NO: 25 - RNA, 8500 nt, Homo sapiens
  source 1..8500: mol_type = other RNA
ggccacagcctgcctactgtcacccgcctctcccgcgcgcagatacacgcccccgcctccgtgggcacaaaggcagcgctgctggggaactcgggggaacgcgcacgtgggaaccgccgcagctccacactccaggtacttcttccaaggacctaggtctctcgcccatcggaaagaaaataattctttcaagaagatcagggacaactgatttgaagtctactctgtgcttctaaatccccaattctgctgaaagtgagataccctagagccctagagccccagcagcacccagccaaacccacctccaccatgggggccatgactcagctgttggcaggtgtctttcttgctttccttgccctcgctaccgaaggtggggtcctcaagaaagtcatccggcacaagcgacagagtggggtgaacgccaccctgccagaagagaaccagccagtggtgtttaaccacgtttacaacatcaagctgccagtgggatcccagtgttcggtggatctggagtcagccagtggggagaaagacctggcaccgccttcagagcccagcgaaagctttcaggagcacacagtggatggggaaaaccagattgtcttcacacatcgcatcaacatcccccgccgggcctgtggctgtgccgcagcccctgatgttaaggagctgctgagcagactggaggagctggagaacctggtgtcttccctgagggagcaatgtactgcaggagcaggctgctgtctccagcctgccacaggccgcttggacaccaggcccttctgtagcggtcggggcaacttcagcactgaaggatgtggctgtgtctgcgaacctggctggaaaggccccaactgctctgagcccgaatgtccaggcaactgtcaccttcgaggccggtgcattgatgggcagtgcatctgtgacgacggcttcacgggcgaggactgcagccagctggcttgccccagcgactgcaatgaccagggcaagtgcgtaaatggagtctgcatctgtttcgaaggctacgccggggctgactgcagccgtgaaatctgcccagtgccctgcagtgaggagcacggcacatgtgtagatggcttgtgtgtgtgccacgatggctttgcaggcgatgactgcaacaagcctctgtgtctcaacaattgctacaaccgtggacgatgcgtggagaatgagtgcgtgtgtgatgagggtttcacgggcgaagactgcagtgagctcatctgccccaatgactgcttcgaccggggccgctgcatcaatggcacctgctactgcgaagaaggcttcacaggtgaagactgcgggaaacccacctgcccacatgcctgccacacccagggccggtgtgaggaggggcagtgtgtatgtgatgagggctttgccggtgtggactgcagcgagaagaggtgtcctgctgactgtcacaatcgtggccgctgtgtagacgggcggtgtgagtgtgatgatggtttcactggagctgactgtggggagctcaagtgtcccaatggctgcagtggccatggccgctgtgtcaatgggcagtgtgtgtgtgatgagggctatactggggaggactgcagccagctacggtgccccaatgactgtcacagtcggggccgctgtgtcgagggcaaatgtgtatgtgagcaaggcttcaagggctatgactgcagtgacatgagctgccctaatgactgtcaccagcacggccgctgtgtgaatggcatgtgtgtttgtgatgacggctacacaggggaagactgccgggatcgccaatgccccagggactgcagcaacaggggcctctgtgtggacggacagtgcgtctgtgaggacggcttcaccggccctgactgtgcagaactctcctgtccaaatgactgccatggccagggtcgctgtgtgaatgggcagtgcgtgtgccatgaaggatttatgggcaaagactgcaaggagcaaagatgtcccagtgactgtcatggccagggccgctgcgtggacggccagtgcatctgccacgagggcttcacaggcctggactgtggccagcactcctgccccagtgactgcaacaacttaggacaatgcgtctcgggccgctgcatctgcaacgagggctacagcggagaagactgctcagaggtgtctcctcccaaagacctcgttgtgacagaagtgacggaagagacggtcaacctggcctgggacaatgagatgcgggtcacagagtaccttgtcgtgtacacgcccacccacgagggtggtctggaaatgcagttccgtgtgcctggggaccagacgtccaccatcatccaggagctggagcctggtgtggagtactttatccgtgtatttgccatcctggagaacaagaagagcattcctgtcagcgccagggtggccacgtacttacctgcacctgaaggcctgaaattcaagtccatcaaggagacatctgtggaagtggagtgggatcctctagacattgcttttgaaacctgggagatcatcttccggaatatgaataaagaagatgagggagagatcaccaaaagcctgaggaggccagagacctcttaccggcaaactggtctagctcctgggcaagagtatgagatatctctgcacatagtgaaaaacaatacccggggccctggcctgaagagggtgaccaccacacgcttggatgcccccagccagatcgaggtgaaagatgtcacagacaccactgccttgatcacctggttcaagcccctggctgagatcgatggcattgagctgacctacggcatcaaagacgtgccaggagaccgtaccaccatcgatctcacagaggacgagaaccagtactccatcgggaacctgaagcctgacactgagtacgaggtgtccctcatctcccgcagaggtgacatgtcaagcaacccagccaaagagaccttcacaacaggcctcgatgctcccaggaatcttcgacgtgtttcccagacagataacagcatcaccctggaatggaggaatggcaaggcagctattgacagttacagaattaagtatgcccccatctctggaggggaccacgctgaggttgatgttccaaagagccaacaagccacaaccaaaaccacactcacaggtctgaggccgggaactgaatatgggattggagtttctgctgtgaaggaagacaaggagagcaatccagcgaccatcaacgcagccacagagttggacacgcccaaggaccttcaggtttctgaaactgcagagaccagcctgaccctgctctggaagacaccgttggccaaatttgaccgctaccgcctcaattacagtctccccacaggccagtgggtgggagtgcagcttccaagaaacaccacttcctatgtcctgagaggcctggaaccaggacaggagtacaatgtcctcctgacagccgagaaaggcagacacaagagcaagcccgcacgtgtgaaggcatccactgaacaagcccctgagctggaaaacctcaccgtgactgaggttggctgggatggcctcagactcaactggaccgcagctgaccaggcctatgagcactttatcattcaggtgcaggaggccaacaaggtggaggcagctcggaacctcaccgtgcctggcagccttcgggctgtggacataccgggcctcaaggctgctacgccttatacagtctccatctatggggtgatccagggctatagaacaccagtgctctctgctgaggcctccacaggggaaactcccaatttgggagaggtcgtggtggccgaggtgggctgggatgccctcaaactcaactggactgctccagaaggggcctatgagtactttttcattcaggtgcaggaggctgacacagtagaggcagcccagaacctcaccgtcccaggaggactgaggtccacagacctgcctgggctcaaagcagccactcattataccatcaccatccgcggggtcactcaggacttcagcacaacccctctctctgttgaagtcttgacagaggaggttccagatatgggaaacctcacagtgaccgaggttagctgggatgctctcagactgaactggaccacgccagatggaacctatgaccagtttactattcaggtccaggaggctgaccaggtggaagaggctcacaatctcacggttcctggcagcctgcgttccatggaaatcccaggcctcagggctggcactccttacacagtcaccctgcacggcgaggtcaggggccacagcactcgaccccttgctgtagaggtcgtcacagaggatctcccacagctgggagatttagccgtgtctgaggttggctgggatggcctcagactcaactggaccgcagctgacaatgcctatgagcactttgtcattcaggtgcaggaggtcaacaaagtggaggcagcccagaacctcacgttgcctggcagcctcagggctgtggacatcccgggcctcgaggctgccacgccttatagagtctccatctatggggtgatccggggctatagaacaccagtactctctgctgaggcctccacagccaaagaacctgaaattggaaacttaaatgtttctgacataactcccgagagcttcaatctctcctggatggctaccgatgggatcttcgagacctttaccattgaaattattgattccaataggttgctggagactgtggaatataatatctctggtgctgaacgaactgcccatatctcagggctaccccctagtactgattttattgtctacctctctggacttgctcccagcatccggaccaaaaccatcagtgccacagccacgacagaggccctgccccttctggaaaacctaaccatttccgacattaatccctacgggttcacagtttcctggatggcatcggagaatgcctttgacagctttctagtaacggtggtggattctgggaagctgctggacccccaggaattcacactttcaggaacccagaggaagctggagcttagaggcctcataactggcattggctatgaggttatggtctctggcttcacccaagggcatcaaaccaagcccttgagggctgagattgttacagaagccgaaccggaagttgacaaccttctggtttcagatgccaccccagacggtttccgtctgtcctggacagctgatgaaggggtcttcgacaattttgttctcaaaatcagagataccaaaaagcagtctgagccactggaaataaccctacttgcccccgaacgtaccagggacataacaggtctcagagaggctactgaatacgaaattgaactctatggaataagcaaaggaaggcgatcccagacagtcagtgctatagcaacaacagccatgggctccccaaaggaagtcattttctcagacatcactgaaaattcggctactgtcagctggagggcacccacagcccaagtggagagcttccggattacctatgtgcccattacaggaggtacaccctccatggtaactgtggacggaaccaagactcagaccaggctggtgaaactcatacctggcgtggagtaccttgtcagcatcatcgccatgaagggctttgaggaaagtgaacctgtctcagggtcattcaccacagctctggatggcccatctggcctggtgacagccaacatcactgactcagaagccttggccaggtggcagccagccattgccactgtggacagttatgtcatctcctacacaggcgagaaagtgccagaaattacacgcacggtgtccgggaacacagtggagtatgctctgaccgacctcgagcctgccacggaatacacactgagaatctttgcagagaaagggccccagaagagctcaaccatcactgccaagttcacaacagacctcgattctccaagagacttgactgctactgaggttcagtcggaaactgccctccttacctggcgacccccccgggcatcagtcaccggttacctgctggtctatgaatcagtggatggcacagtcaaggaagtcattgtgggtccagataccacctcctacagcctggcagacctgagcccatccacccactacacagccaagatccaggcactcaatgggcccctgaggagcaatatgatccagaccatcttcaccacaattggactcctgtaccccttccccaaggactgctcccaagcaatgctgaatggagacacgacctctggcctctacaccatttatctgaatggtgataaggctgaggcgctggaagtcttctgtgacatgacctctgatgggggtggatggattgtgttcctgagacgcaaaaacggacgcgagaacttctaccaaaactggaaggcatatgctgctggatttggggaccgcagagaagaattctggcttgggctggacaacctgaacaaaatcacagcccaggggcagtacgagctccgggtggacctgcgggaccatggggagacagcctttgctgtctatgacaagttcagcgtgggagatgccaagactcgctacaagctgaaggtggaggggtacagtgggacagcaggtgactccatggcctaccacaatggcagatccttctccacctttgacaaggacacagattcagccatcaccaactgtgctctgtcctacaaaggggctttctggtacaggaactgtcaccgtgtcaacctgatggggagatatggggacaataaccacagtcagggcgttaactggttccactggaagggccacgaacactcaatccagtttgctgagatgaagctgagaccaagcaacttcagaaatcttgaaggcaggcgcaaacgggcataaattccagggaccactgggtgagagaggaataaggcccagagcgaggaaaggattttaccaaagcatcaatacaaccagcccaaccatcggtccacacctgggcatttggtgagagtcaaagctgaccatggatccctggggccaacggcaacagcatgggcctcacctcctctgtgatttctttctttgcaccaaagacatcagtctccaacatgtttctgttttgttgtttgattcagcaaaaatctcccagtgacaacatcgcaatagttttttacttctcttaggtggctctgggaatgggagaggggtaggatgtacaggggtagtttgttttagaaccagccgtattttacatgaagctgtataattaattgtcattatttttgttagcaaagattaaatgtgtcattggaagccatccctttttttacatttcatacaacagaaaccagaaaagcaatactgtttccattttaaggatatgattaatattattaatataataatgatgatgatgatgatgaaaactaaggatttttcaagagatctttctttccaaaacatttctggacagtacctgattgtattttttttttaaataaaagcacaagtacttttgagtttgttattttgctttgaattgttgagtctgaatttcaccaaagccaatcatttgaacaaagcggggaatgttgggataggaaaggtaagtagggatagtggtcaagtgggaggggtggaaaggagactaaagactgggagagagggaagcactttttttaaataaagttgaacacacttgggaaaagcttacaggccaggcctgtaatcccaacactttgggaggccaaggtgggaggatagcttaaccccaggagtttgagaccagcctgagcaacatagtgagaacttgtctctacagaaaaaaaaaaaaaaaaaaatttaattaggcaagcgtggtagtgcgcacctgtcgtcccagctactcaggaggctgaggtaggaaaatcactggagcccaggagttagaggttacagtgagctatgatcacactactgcactccagcctgggcaacagagggagaccctgtctctaaataaaaaaagaaaagaaaaaaaaagcttacaacttgagattcagcatcttgctcagtatttccaagactaatagattatggtttaaaagatgcttttatactcattttctaatgcaactcctagaaactctatgatatagttgaggtaagtattgttaccacacatgggctaagatccccagaggcagactgcctgagttcaattcttggctccaccattcccaagttccctaacctctctatgcctcagtttcctcttctgtaaagtagggacactcatacttctcatttcagaacatttttgtgaagaataaattatgttatccatttgaggcccttagaatggtacccggtgtatattaagtgctagtacatgttagctatcatcattatcactttatatgagatggactggggttcatagaaacccaatgacttgattgtggctactactcaataaataatagaatttggatttaaa